(19) United States
(12) Patent Application Publication
(10) Pub. No.: US 2014/0243401 A1
(43) Pub. Date: Aug. 28, 2014

(54) METHODS OF SCREENING COMPOUNDS THAT ARE CYTOTOXIC TO TUMOR CELLS AND METHODS OF TREATING TUMOR CELLS USING SUCH COMPOUND

(71) Applicant: Gabrielle Ronnett, Lutherville, MD (US)

(72) Inventors: Frank Kuhajda, Lutherville, MD (US); Gabrielle Ronnett, Lutherville, MD (US); Craig Townsend, Baltimore, MD (US)

(73) Assignee: The Johns Hopkins University, Baltimore, MD (US)

(21) Appl. No.: 13/989,731

(22) PCT Filed: Nov. 23, 2011
(86) PCT No.: PCT/US11/62132

§ 371 Date: Jul. 24, 2013

Related U.S. Application Data
(60) Provisional application No. 61/416,833, filed on Nov. 24, 2010.

(51) Int. Cl. G01N 33/50

(52) U.S. Cl. CPC G01N 33/5011
USPC 514/445; 435/32; 435/375

(57) ABSTRACT

The invention relates to methods of screening to find compounds that are cytotoxic to tumor cells and methods of treating tumor cells using these compounds. In particular, the invention relates to methods of screening for compounds that inhibit mammalian mitochondrial fatty acid synthase (mmFAS) and methods of treating tumor cells using mmFAS inhibitors. This invention also provides methods for inhibiting or preventing cancer cell survival by the administration of mitochondrial fatty acid synthase (FAS) inhibitors. Specifically, this invention describes a method for prohibiting or delaying the development of cancer, the growth of cancer or invasion of cancer from pre-malignant (noninvasive) lesions, or metastasis of cancer based upon the findings that this method compromises energy balance in cancer cells, in turn compromising their basic functions and causing their cell death. Compositions of matter containing mitochondrial FAS inhibition activity are also provided, as well as applications based upon the requisite role of mitochondrial FAS in cancer cell homeostasis.

### BACKGROUND OF THE INVENTION

(a) Field of the Invention

The invention relates to methods of screening to find compounds that are cytotoxic to tumor cells and methods of treating tumor cells using these compounds. In particular, the invention relates to methods of screening for compounds that inhibit mammalian mitochondrial fatty acid synthase (mmFAS) and methods of treating tumor cells using mmFAS inhibitors.

(b) Description of the Related Art

Fatty acids have three primary roles in the physiology of cells. First, they are the building blocks of biological membranes. Second, fatty acid derivatives serve as hormones and intracellular messengers. Third, fatty acids are fuel molecules that can be stored in adipose tissue as triacylglycerols, which are also known as neutral fats.

Fatty Acid Synthase Enzyme Systems

A key enzyme in the de novo synthesis of fatty acids is fatty acid synthase (FAS) or the FAS enzyme system. FAS is a multistep enzyme reaction pathway (Toomey and Wakil, 1966). Although FAS displays significant variations in molecular structures when isolated from different sources, the reaction mechanism for de novo fatty acid synthase displays some overall similarities in all biological systems thus far examined (Smith, 1994; Smith et al., 2003). FAS is a classic example of an iterative multienzyme, performing several successive cycles of distinct reaction sequence in combination with specific initiation and termination reactions (Lynen, 1980; Toomey and Wakil, 1966; Vagelos, 1969). There are 7 distinct enzymes involved in the FAS catalyzed synthesis of fatty acids. The individual FAS component activities include acyltransferase (AC) malonyl/acetyl- or malonyl/palmitoyl-transacylase (AT, M/PT), ketoacyl synthase (KS), ketoacyl reductase (KR), dehydratase (DH), enoyl reductase (ER), acyl carrier protein (ACP), and thioesterase (TE) (Wakil). There are two general classes of FAS enzymes (Schweizer, 1996; Schweizer and Hofmann, 2004).

The first and to date best characterized class is the highly integrated type I FAS multi-enzymes, which most commonly contain all the catalytic activities of the reaction sequence as distinct functional domains on a single polypeptide chain (Lynen, 1980; Rangan, 2003; Schweizer and Hofmann, 2004). Type I FAS enzymes exist as homodimers and in some less common cases as two different multifunctional proteins of comparable size (Smith, 1994; Smith et al., 2003). Type I multi-enzymes are usually found in the eukaryotic cytoplasm, and in some prokaryotes, such as the mycolic acid producing subgroup of Actinomycetales (Bloch and Vance, 1977; Seyama, 1987). The type I systems may be further divided on the basis of domain organization and subunit stoichiometry (Schweizer and Hofmann, 2004). Microbial type I FAS are hexamers that contain AC-ER-DH-MPT/ACP-KR-KS, as either an alpha6-beta6 (fungi) or as alpha6 (bacteria) oligomers (Type Ia). In contrast, animal FAS Type I contains alpha2 dimers with the domain sequence KS-AT-DH-ER-KR-ACP-TE (Type Ib). FAS may also be considered the ancestors of most members of the large family of polyketide synthases (PKSs)

Type I FAS has been proposed for cancer therapy because it acts only within the pathway to fatty acids, but it is not implicated in other cellular functions. (See U.S. Pat. Nos. 5,759,837 and 5,981,575, which are hereby incorporated by reference). Therefore, inhibition of type I FAS is not likely to affect normal cells. Type I FAS inhibitors are known in the art. (See, e.g., U.S. Pat. Nos. 5,759,837 and 5,981,575; Rivkin et al., Bioorganic & Medicinal Chemistry Letters 16: 4620-4623, 2006).

In contrast, the second class of FAS systems is represented by the dissociated Type II FAS enzyme system, which is found in most bacteria, as well as in mitochondria and chloroplasts. What distinguishes these Type II enzymes is that the individual FAS reactions are performed by independent and distinct proteins that are encoded by a series of separate genes. The studies of Type II FAS began with work on bacteria, yeast, and plant chloroplasts (White et al., 2005). These Type II FAS enzymes have been identified from a number of sources, and have been viewed as possible targets for antibiotic development, but as of yet not for other purposes (Schweizer and Hofmann, 2004).

Due to the existence of discrete proteins for each of the reactions attributed to the dissociated Type II FAS systems, identification of these components has been difficult. Yeasts have served as a model for investigation of the mitochondrial Type II FAS and its mammalian counterparts. Mitochondrial fatty acid synthesis and the existence of a type II ACP were described by Brody and coworkers in Neurospora crassa (Brody and Mikolajczyk, 1988; Brody et al., 1997). Since then, a mitochondrial form of ACP has been demonstrated to be present in other eukaryotes and most of the other FAS components have been characterized in yeast and humans. Saccharomyces cerevisia has also served as a model for studies of Type II FAS enzymes, (Harington et al., 1993). Independently, a S. cervisiae gene with significant sequence similarity at the amino acid level to bacterial FAS was identified by Slonimski (Harington et al., 1993).

This pathway produces an octanoyl-ACP substrate for lipoic acid synthesis, but several pieces of evidence indicate that it can produce longer chain fatty acids. Inactivation of mitochondrial FAS II in yeast results in respiratory deficiency and loss of cytochromes. In the yeast S. cerevisiae (Hiltunen et al., 2010a; Hiltunen et al., 2009), deletion of any member of the mitochondrial FAS pathway leads to a respiratory deficient phenotype, lack of cytochromes, and a decrease in lipoic acid, and small rudimentary mitochondria, indicating that this pathway is essential for mitochondrial function (Schonauer et al., 2008). Subsequent to work in yeast, several human FAS type II homologs were isolated and identified.

Mitochondiral Fatty Acid Synthase

We now appreciate that the de novo synthesis of fatty acids in eukaryotes can take place in two subcellular compartments, the cytoplasm, where FAS Type I resides, and in the mitochondria, where FAS Type II is found (Schweizer, 1996). This non-cytoplasmic source for fatty acids is hypothesized to serve a variety of specialized functions (Schweizer, 1996). Mitochondrial fatty acid synthesis pathway follows the bacterial type II FAS mode, with separate polypeptides carrying out the individual reactions (White et al., 2005). The activities of mitochondrial FAS are catalyzed by soluble enzymes, and the pathway thus resembles its prokaryotic counterpart (Hiltunen et al., 2009).

Most of the eukaryotic FAS II enzymes show amino acid similarities to their prokaryotic counterparts, and hence database mining has been used for identification of mitochondrial equivalents in eukaryotes. To date, ACP, phosphopantotheine transferase (PPTase) (Joshi et al., 2003), malonyl transferase (MCAT), beta-ketoacyl-ACP synthase (KAS), 3-hydroxyacyl-thioester dehydratase (HTD), enoyl thioester reductase (ETR) of human mitochondrial FAS II pathway, and 3-ketoacyl reductase (KAR) of the mammalian pathway have been identified (Chen et al., 2009). Among the recently recognized features of the mitochondrial FAS pathway is its ability to synthesize fatty acids in an ACP-dependent manner. Mitochondria contain an ACP as a structural component of the membrane bound mitochondrial respiratory complex I, but it was later shown that mammalian mitochondria also contain a soluble form ACP (Cronan et al., 2005).

Metabolic Opportunities in Cancer Therapy

Since the time of Otto Warburg (Warburg, 1956a, b), it has been appreciated that cancer cells operate and may rely upon metabolic pathways that are different from those found in normal cells, or may exploit these pathways for other benefits as needed for growth in hostile environments, such as hypoxia and hypoglycemia, as found within the cancer microenvironment. Many investigators are studying the role of different isoforms of glycolic enzymes in cancer cells in the regulation at key branch points between glycolysis and the citric acid cycle as offering opportunities for cancer chemotherapy based on the requirement of cancer cells to obtain energy equivalence in the setting of relative hypoxia and hypoglycemia.

Alterations in cellular bioenergetics are an emerging hallmark of cancer. Recently, several cancer management therapies have been proposed the target tumor cell metabolism and mitochondria (Pathania et al., 2009). The main targets of interest involve glycolic inhibitors, which serve as classical examples of cancer metabolism targeting agents. Several TCA cycle an oxidative phosphorylation inhibitors are being tested for their anticancer capacity. Recently, agents targeting the PDC/PDK (pyruvate dehydrogenase complex/pyruvate dehydrogenase kinase) interactions are being studied for reversal of the Warburg effect. Additionally, investigators have targeted the apoptotic regulatory machinery of mitochondria as another potential anticancer therapy. Also, oxidative phosphorylation uncouplers and mitochondrial redox modulators are under investigation. However, manipulation of mitochondrial FAS activities or pathways has not been entertained. The main interest in altering lipid metabolism appears to be as a method for altering the incorporation of lipids into membranes, or reducing lipids that may be required for protein modifications. Thus far, there is no report of using the mitochondrial fatty acid synthase system as a target for cancer therapy.

There remains a need in the art for developing compounds that are cytotoxic to cancer cells but non-toxic to non-cancerous cells. There is also a need in the art for methods of treating cancer using such compounds.

### SUMMARY OF THE INVENTION

The invention addresses these needs. Indeed, the invention provides for various methods of screening compounds that are cytotoxic to tumor cells and methods of treating tumor cells using these compounds.

In one aspect, the invention provides a method for identifying compounds likely to inhibit growth of tumors where the method comprises (a) performing an assay that measures a biological activity selected from the group consisting of (i) mmFAS activity, (ii) Type I FAS activity, (iii) mitochondrial transmembrane potential, and (iv) cell viability in the presence and absence of a test compound; and (b) determining whether the presence of the test compound inhibits the activity.

In one mode, the assay measures mmFAS activity. Typically, this activity is measured in the mitochondria. MmFAS is typically determined by rate of incorporation of malonate into lipids, optionally using radioactivity. In a preferred mode, the method also measures Type I FAS, typically by determining rate of incorporation of acetate into lipids. Preferably, the test compound exhibits an IC50 for inhibition of mmFAS that is lower than its IC50 for inhibition of Type I FAS activity. More preferably, the ratio of IC50 for inhibition of Type I FAS to the IC50 for inhibition of mmFAS is at least ten.

In another mode, the assay measures mitochondrial transmembrane potential, wherein the transmembrane potential is optionally measured by transmembrane accumulation of a fluorescent dye. In yet another mode, the assay measures cell viability. Preferably, the effect of the test compound is determined in more than one of the assays described herein; more preferably in all of the assays described herein. Optionally, the suitability of the test compound may be confirmed by determining whether the test compound reduces the growth of one or more xenograft tumors without killing the host.

Compounds that demonstrate effective inhibition of one or more of the biological activities described herein are likely to be cytotoxic to various cancer cells. Effective inhibition of more than one of these biological activities indicates increased likelihood of cytotoxicity. Such cytotoxic compounds may be used to treat malignancies as described herein.

The present invention provides a method for inhibiting cancer development or cancer cell proliferation or growth by the administration of mitochondrial FAS inhibitors. Compositions containing FAS inhibitors are also provided.

In one mode, the present invention also provides a method of inhibiting the growth of cancer cells, where the method comprises (a) selecting tumor cells which overexpress mammalian mitochondrial FAS, and (b) administering a compound in an amount sufficient to inhibit the growth of the tumor cells, where the compound has a structure represented by Formula I:

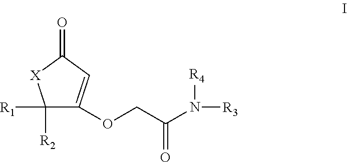

where X is comprised of a heteroatom which may be selected from any one of O, S, or NR, where R is H, alkyl, alkenyl, aryl, arylaklyl, or alkylaryl. R₁ and R₂ are independently selected from H, C1-C20 alkyl, cycloalkyl, alkenyl, aryl, arylalkyl, or alkylaryl. R₃ and R₄ are independently either a hydrogen atom or are members of a substituted or unsubstituted ring having 4-6 carbon atoms, or where if neither R₃ and R₄ is a hydrogen, then they together form an optionally substituted ring structure having 4-6 carbon atoms.

In another mode, the present invention provides a method of inhibiting cancer cell proliferation comprising exposing cancer cells to a compound which exhibits an IC₅₀ for inhibition of mammalian mitochondrial fatty acid synthase (mmFAS) that is lower than its IC₅₀ for inhibition of Type I fatty acid synthase (FAS) activity, more preferably where the IC₅₀ for inhibition of Type I FAS is at least ten fold higher than the IC₅₀ for mmFAS.

In yet another mode, the invention provides a method of inhibiting cancer cell proliferation comprising exposing cancer cells an inhibitor of mmFAS in an amount sufficient to interrupt the metabolic balance of cells in the tumor, typically by reducing cellular ATP level, reducing mitochondrial membrane polarization, and/or increasing AMP-activated protein kinase phosphorylation.

In a preferred embodiment, the invention provides a method of inhibiting tumor proliferation comprising administering an inhibitor of mmFAS in an amount cytotoxic to tumor cells under restricted levels of oxygenation and/or nutrient levels. In one mode, this preferred invention provides a method of inhibiting tumor proliferation comprising administering an inhibitor of mmFAS to a subject having a solid tumor in an amount sufficient to achieve systemic concentration of the inhibitor which is cytotoxic to cells on the outside of the tumor, whereby a lower concentration of the inhibitor on the interior of the tumor is still cytotoxic to interior cells.

Compounds suitable for use in any of the therapeutic methods of this invention include an mmFAS inhibitor having a structure represented by Formula I:

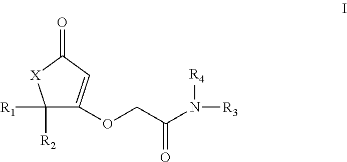

wherein X is comprised of a heteroatom which may be selected from any one of O, S, or NR, where R is H, alkyl, alkenyl, aryl, arylaklyl, or alkylaryl. R₁ and R₂ are independently selected from H, C1-C20 alkyl, cycloalkyl, alkenyl, aryl, arylalkyl, or alkylaryl. R₃ and R₄ are independently either a hydrogen atom or are members of a substituted or unsubstituted ring having 4-6 carbon atoms, or where if neither R3 and R4 is a hydrogen, then they together form an optionally substituted ring structure having 4-6 carbon atoms. Preferably, the fatty acid synthase inhibitor is a compound having the formula:

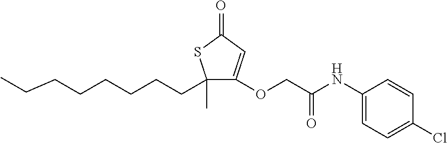 [SMILES: CCCCCCCCC1(C)SC(=O)C=C1OCC(=O)Nc1ccc(Cl)cc1]

Compounds suitable for use in any of the therapeutic methods of this invention also include an mmFAS inhibitor which s a diphenyl ether, such as triclosan.

Preferably the methods of this invention may be applied to cancer cells in a solid tumor or a pre-cancerous lesion. In particular, the pre-cancerous or cancerous lesion may be in a tissue type in human consisting of breast, prostate, colon, lung, stomach, mouth, bile duct, ovarian, brain, or liver.

In another embodiment, this invention provides a method for determining the responsiveness of a pre-cancerous or cancerous lesion to mitochondrial FAS inhibitors using serum testing or tissue immunohistochemistry to evaluate for the presence of mitochondrial FAS. This invention also provides a method for screening the efficacy of human tumors to mitochondrial FAS inhibitors by assaying tumor tissue directly for it sensitivity against mitochondrial FAS inhibitors. Alternatively, this invention provides a method for screening the susceptibility of human tumors to mitochondrial FAS inhibitors by assaying for over-expression of mitochondrial FAS, either in the tumor tissue directly or in the tumor host. Methods of detecting one or more components of the mitochondrial FAS enzyme complex are known to those of skill in the art.

In another embodiment, the present invention provides a method for identifying pharmacological compositions containing pharmacologically acceptable additives by screening the efficacy of these pharmacological compositions in inhibiting activities of mitochondrial FAS.

The inventors have discovered that inhibitors of mammalian mitochondrial fatty acid synthase (mmFAS inhibitors) are cytotoxic to various cancer cells. Based on this and other discoveries, the inventors have developed the present methods of screening to find compounds that are cytotoxic to tumor cells and methods of treating carcinoma patients by administering inhibitors of mammalian mitochondrial fatty acid synthesis to reduce tumor burden in the patients.

### BRIEF DESCRIPTION OF THE DRAWINGS

FIG. 1 A illustrates the dose-response curve of ¹⁴C-acetate incorporation into lipid in the presence of increasing concentrations of C 31 in MCF 7 cells.

FIG. 1 B illustrates the dose-response curve of ¹⁴C-acetate incorporation into lipids in the presence of increasing concentrations of Riv7d in MCF 7 cells.

FIG. 2 illustrates the effect of vehicle or control, C31, or Riv7d on ¹⁴C-malonate incorporation in isolated mitochondria from MCF 7 cells.

FIG. 3 illustrates the effect of vehicle or control, C31, or Riv7d on ¹⁴C-malonate incorporation in isolated mitochondria from MCF 7 cells. Triclosan (TCS), a bacterial FAS inhibitor, is used as a positive control.

FIG. 4 illustrates the effects of increasing concentrations of C31 (A) and Riv7d (B) on XTT assay of MCF7 cells and the effects of C31 dosing or Riv7d on clonogenic assays (C).

FIG. 5 illustrates the effects of C31 or Riv7d on ATP levels in MCF 7 cells.

FIG. 6 illustrates the effect of vehicle, C31, or Riv7d on JC-1 staining of MCF 7 cells.

FIG. 7 illustrates visually the effects of vehicle control, C31, or Riv7d on fluorescence emission used to calculate the results of the JC-1 assay, and thus these compounds effects on mitochondrial function.

FIG. 8 illustrates the effects of vehicle (DMSO), C31, or Riv7d on AMPK activation.

FIG. 9 A illustrates the effect of varying glucose and oxygen concentrations on the effects of C31 on XTT assays of MCF 7 cells.

FIG. 9 B illustrates the effect of varying glucose and oxygen concentrations on the effects of Riv7d on XTT assays of MCF 7 cells.

FIG. 10 A illustrates the effect of varying glucose and oxygen concentrations on the effects of C31 on ATP levels of MCF 7 cells.

FIG. 10 B illustrates the effect of varying glucose and oxygen concentrations on the effects of Riv7d on ATP levels of MCF 7 cells.

FIG. 11 illustrates the effects of varying glucose and oxygen concentrations on the effects of vehicle control, C31, or Riv7d on JC-1 staining of MCF 7 cells.

FIG. 12 A illustrates the effects of varying oxygen and glucose concentrations on AMPK activation over time in the presence of C31 in MCF 7 cells.

FIG. 12 B illustrates the effects of varying oxygen and glucose concentrations on AMPK activation over time in the presence of C31 in MCF 7 cells.

FIG. 12 C illustrates the effects of varying oxygen and glucose concentrations on AMPK activation over time in the presence of C31 in MCF 7 cells.

FIG. 12 D illustrates the effects of varying oxygen and glucose concentrations on AMPK activation over time in the presence of C31 in MCF 7 cells.

FIG. 12 E illustrates the quantification of the effects of varying glucose and oxygen concentrations on the effects of C31 on AMPK activation.

FIG. 13 A illustrates the effects of varying oxygen and glucose concentrations on AMPK activation over time in the presence of Riv7d in MCF 7 cells.

FIG. 13 B illustrates the effects of varying oxygen and glucose concentrations on AMPK activation over time in the presence of Riv7d in MCF 7 cells.

FIG. 13 illustrates the effects of varying oxygen and glucose concentrations on AMPK activation over time in the presence of Riv7d in MCF 7 cells.

FIG. 13 D illustrates the effects of varying oxygen and glucose concentrations on AMPK activation over time in the presence of Riv7d in MCF 7 cells.

FIG. 13 E illustrates the quantification of the effects of varying glucose and oxygen concentrations on the effects of C31 on AMPK activation.

FIG. 14 illustrates the effects of varying oxygen and glucose concentrations on the incorporation of ¹⁴C acetate into lipids in MCF 7 cells treated with C31.

Figure X1 shows JC-1 staining of SKOV3 cells in the presence of C31 (also referred to as FSG31). There is a depolarization of the mitochondria in the SKOV3 human ovarian cancer cells which is dose-dependent with C31 concentration. The left panels show granular red-staining indicating mitochondria with intact membrane potential is brightest in control, and decreases with increasing concentrations of C31. Conversely, the right panels show diffuse cytoplasmic green-staining which indicates mitochondrial depolarization, is dimmest in control and brightest at the highest concentration of C31 tested. Drug was solubilized in DMSO and applied for 30 min prior to staining.

Figure X2 shows JC-1 staining of SKOV3 cells in the presence of C144 (also referred to as FSG144). Similar to C31, there is a depolarization of the mitochondria in the SKOV3 human ovarian cancer cells which is dose-dependent with C144 concentration. The left panels show granular red-staining indicating mitochondria with intact membrane potential is brightest in control, and decreases with increasing concentrations of C144. Conversely, the right panels show the diffuse cytoplasmic green-staining which indicates mitochondrial depolarization, is dimmest in control and brightest at the highest concentration of C144 tested. Drug was solubilized in DMSO and applied for 30 min prior to staining.

Figure X3 shows JC-1 staining of SKOV3 cells in the presence of C145 (also referred to as FSG145). Similar to both C31 and C144, there is a depolarization of the mitochondria in the SKOV3 human ovarian cancer cells which is dose-dependent with C145 concentration. The left panels show granular red-staining indicating mitochondria with intact membrane potential is brightest in control, and decreases with increasing concentrations of C145. Conversely, the right panels show the diffuse cytoplasmic green-staining which indicates mitochondrial depolarization, is dimmest in control and brightest at the highest concentration of C145 tested. Drug was solubilized in DMSO and applied for 30 min prior to staining.

### DETAILED DESCRIPTION OF THE EMBODIMENTS

This invention provides a method for inhibiting or preventing cancer cell survival by the administration of mitochondrial fatty acid synthase (FAS) inhibitors. Specifically, this invention describes a method for prohibiting or delaying the development of cancer, the growth of cancer or invasion of cancer from pre-malignant (noninvasive) lesions, or metastasis of cancer based upon the findings that this method compromises energy balance in cancer cells, in turn compromising their basic functions and causing their cell death. Compositions of matter containing mitochondrial FAS inhibition activity are also provided, as well as applications based upon the requisite role of mitochondrial FAS in cancer cell homeostasis.

Regulatory Roles of Mitochondrial Fatty Acid Synthase System

The reason why eukaryotes have maintained a bacterial type FAS in their mitochondria in addition to the classic cytoplasmic FAS is unclear. Except for octanoic acid, which is the direct precursor for lipoic acid synthesis, other end products and functions of the mitochondrial FAS pathway are largely unknown. Interestingly, in yeast respiratory competence is dependent on the ability of mitochondria to synthesize fats. Many new aspects of cellular homeostasis have been found to involve mitochondrial morphology maintenance, fusion and fission events, mitochondrial-nuclear crosstalk, mitochondrial DNA replication, transcription and translation and regulation, the role of mitochondria in apoptosis, are but a few of the biological roles of mitochondria (Hiltunen et al., 2010a). Neither the range of fatty acids produced by the mitochondria nor their roles in cellular metabolism are understood. The pathway may be the sole source of octanoic acid precursor required for the production the lipoic acid cofactor essential for PDH, alpha-ketoglutarate dehydrogenase, and the glycine cleavage system (Brody et al., 1997; Jordan and Cronan, 1997; Schonauer et al., 2008; Witkowski et al., 2007). Thus, mitochondrial FAS may play an as yet undiscovered regulatory role.

An intersection of two pathways frequently provides a point of control of metabolic fluxes. Thus, the present inventors hypothesized that fatty acid metabolic pathways, which sit at the intersection of energy efficiency and energy surplus, may provide an opportunity to alter the perception of energy balance by pharmacological modulation principal enzymes involved in fatty acid metabolism (Kuhajda, 2008). Recently, links between mitochondrial FAS and RNA processing have been identified (Autio et al., 2008; Schonauer et al., 2008). Hiltunen similarly hypothesized that the intersection of the mitochondrial FAS II pathway with RNA processing had been maintained through evolution as a means a regulating mitochondrial function relative to nutritional state of the cell (Hiltunen et al., 2010a; Hiltunen et al., 2009). In yeast, the mitochondrial FAS II pathway controls the maturation or activity mitochondrial RNase P, which cleaves the 5′ and mitochondrial precursors of tRNAs. In yeast, defects in mitochondrial FAS result in inefficient RNase P cleavage in the organelle. In humans as well as in other vertebrates, a nuclear bicistronic mRNA encodes both the RPP14 subunit of RNase P and 3-hydroxyacyl-ACP dehydratase (HDT 2) In the FAS II Pathway. The present invention describes a new function for mitochondrial FAS in cancer cells that can be utilized to compromise cell function and obtain a chemotherapeutic effect.

The intersection of mitochondrial FAS and RNA metabolism in both systems provide a novel mechanism for the coordination of intermediary metabolism in eukaryotic cells. Thus, subsequent to the initial effects resulting from inhibition of mitochondrial FAS activity, which may affect lipoic acid levels or the ambient levels of longer chain fatty acids in mitochondria, inhibition of mitochondrial fatty acid synthesis may have downstream effects on substrate level reduction/oxidation (redox) or a reduction in fatty acid levels that are required for the additional source of ATP their oxidation may generate in the setting of uncontrolled growth in the extreme environment of transformed cells.

Lipoic acid is generally regarded as a nutritional requirement in mammals (Challem, 1999). A number of pharmacological roles for lipoic acid, ranging from the treatment of the consequences of diabetes to mood disorders through the relief of oxidative stress, have been described (Maczurek et al., 2008; Pepe et al., 2008; Singh and Jialal, 2008; Soczynska et al., 2008). There is a large body of evidence confirming that lipoic acid is a powerful antioxidant with vitamin-like features in humans. Mammals rely on lipoic acid-dependent enzyme complexes for mitochondrial and respiratory functions. The effects of a knockout mutation of mouse lipoic acid synthase on embryonic growth cannot be reversed by supplementary feeding of lipoic acid to the mother. The possibility posed by the inventors that mitochondrially synthesized lipoic acid and lipoic acid taken as a nutrient may fulfill two different roles in mammalian metabolism. In addition to octanoic acid, there is evidence that the FAS II pathway also synthesizes longer chain fatty acids. The active sites accept substrates with carbon chains as long as 2 all the way up to 14 to 16 carbons. A physiological function for the longer fatty acids produced by the mitochondrial FAS pathway is unknown.

It is proposed that a positive feedback loop exists in yeast to regulate mitochondrial function in response to pyruvate availability in glucose growing cells. FAS produces octanoic acid, which is the substrate for lipoic acid synthesis. Lipoic acid is attached to a subunit of PDH and is required for the conversion of pyruvate to acetyl-CoA, which feeds into the FAS pathway.

Using standard in vitro growth inhibition assays, the inventors have demonstrated that inhibitors of mmFAS inhibit growth of carcinoma cells, but have little effect on normal human hepatocytes. Indeed, studies with multiple breast, lung, colon, and ovarian carcinoma cell lines, confirm that mmFAS inhibitors inhibit the growth of carcinoma cells. Thus, inhibition of the mammalian mitochondrial fatty acid synthase enzyme can inhibit the growth of cells in tumors. As such, the invention provides for methods of screening for mmFAS inhibitors that are cytotoxic to tumor cells and methods of treating tumor cells using mmFAS inhibitors. It has also been demonstrated that mmFAS inhibitors inhibit the growth of leukemia and multiple myeloma cell lines.

### DEFINITIONS

As used herein, “mmFAS” or “mammalian mitochondrial fatty acid synthase” means the various components of the fatty acid synthase enzyme complex present in the mitochondria of a mammalian cell. This complex comprises a number of discrete polypeptides which are analogous to polypeptides of the Type II FAS observed in bacteria. MmFAS inhibitors are compounds which affect one or more of the component polypeptides of mmFAS, where this effect results in reduction of fatty acid synthesis by mmFAS.

As used herein, the term “overexpression” of an enzyme by a cell line or tissue means that cells have higher enzyme activity than cells of the same type on normal tissue relative to the total protein content of the cells. Studies by the inventors indicate that cancer cells, on a specific activity basis of enzyme activity per milligram of protein, have 2-5× more mmFAS

As used herein, “IC₅₀ for inhibition” means the half maximal inhibitory concentration is a measure of the effectiveness of a compound at inhibiting a biological or biochemical function; it is the concentration at which the biological or biochemical process is inhibited to 50% of its activity. IC₅₀ values for two different compounds are different if the IC₅₀ values measured for each compound differ from each other by more than twice the standard error of the measurement on which the IC₅₀ is based.

As used herein, the term “inhibiting cancer cells” is understood to mean preventing, suppressing, retarding, or delaying cancer cell development, growth, or metastasis, by interfering with the homeostasis of cancer cells and thus triggering apoptosis or other forms of cell death.

As used herein, the term “cancer development” is understood to mean the initial appearance of cancerous cells. By cancerous cells, we mean cells which have properties of unregulated cell growth and proliferation, that may have the ability to invade or compromise adjacent tissues.

As used herein, “cancer cell proliferation” means growth of cancer cells, including replication to generate new cells, or increase in mass or volume size of cancer cells.

As used herein, “tumor proliferation” means the growth, replication to generate new cells, or increase in mass or volume size of cancer cells comprising a tumor mass or metastasis

As used herein, “compromise energy balance in cells” means to affect the metabolism of a cell so that there is a reduction of energy in the form of chemical energy, that is energy as stored in chemical bonds such as ATP, to facilitate anabolic processes in the cell, such as growth, replication, movement, further metabolism, synthesis of organelles or membranes. Effects of compounds which compromise the energy balance of a cell include lowering of total cellular ATP level, depolarizing of cellular membranes, particularly mitochondrial membranes, and frequently cell death.

As used herein, the term “administration” is understood to mean any of the multiple of possible means of administrations commonly used in the art, such as, for example orally, rectally, nasally, parenterally, and the like, wherein parenteral administration includes, for example intravenous, intramuscular, intra-peritoneal, intra-plural, intra-vesicular, intrathecal, subcutaneous, or topical.

Methods of Screening Compounds

The invention provides for a method of screening compounds that are cytotoxic to tumor cells. These methods involve administering a test compound to mammalian (e.g., human) tumor cells that express or overexpress mammalian mitochondrial fatty acid synthase (mmFAS), and determining whether the test compound is cytotoxic to the tumor cells. If the test compound decreases mmFAS activity as compared to the mmFAS activity in the absence of the test compound, then the test compound will be cytotoxic to the tumor cells.

Preferred compounds are mitochondrial FAS inhibitor, which should be understood to mean a synthetic or derived compound which directly inhibits a component or reaction in the pathway that comprises mitochondrial fatty acid synthesis, a pathway that is comprised of distinct sequential reaction steps collectively termed mitochondrial FAS, or equally validly mitochondrial fatty acid synthesis. Direct inhibition means that the inhibitor reduces mitochondrial FAS activity by direct action on the enzyme. While there may be some effect on cytoplasmic FAS, this secondary activity is not the critical or crucial element or the major effect, which leads to inhibition of cancer cell development, growth, or metastasis. An exemplary mitochondrial FAS inhibitor is C31, which is used throughout this specification representatively.

There are several methods known in the art for identification of FAS inhibitors, based upon the ability of compounds to inhibit the enzymatic activity of purified FAS. For example, one can measure the oxidation of NADPH in the presence of malonylCoA, or incorporation of radiolabeled acetate or malonate into lipids. Of the enzymatic steps required for the formation of fatty acids, several may serve as preferred candidates for inhibitor targeting.

A. Measuring Type I Cytosolic FAS and mmFAS Activity

The invention provides for methods that involve determining and/or measuring Type I and mmFAS activity. Methods of measuring Type I FAS activity are known in the art. (See, e.g., U.S. Pat. Nos. 5,759,837 and 5,981,575, which are hereby incorporated by reference in their entirety). For example, Type I FAS activity may be measured in whole cells by monitoring the incorporation of [14C]acetate into total lipids. In cell lysates, Type I FAS activity may be measured by monitoring the incorporation of [14C] from malonyl CoA into total lipids. Alternatively, Type I FAS activity may be determined using purified enzyme with acetyl CoA and malonyl CoA as substrates by measuring incorporation of radioactivity or measuring oxidation of NADPH spectrophotometrically. To test the inhibition of Type I FAS activity, a test compound is added to the reaction mixture.

Mammalian mitochondrial FAS activity may be determined in a variety of ways. For example, [14C]malonate may be added to isolated mitochondria, fatty acids are extracted, and then radioactivity is tested. (See Zhang et al., J. Bio. Chem. 278(41): 40067-40074, 2003; Mikolajczyk, J. and Brody, S., Eur. J. Biochem. 187: 431-437, 1990). The mitochondria are purified using methods known in the art. (See Qiagen's Qproteome Mitochondria Isolation Kit; Methods in Enzymology, Vol. 10, Chapter 11, 1967). To test the inhibition of mmFAS activity, a test compound is added to the reaction mixture. Therefore, a test compound may be screened according the methods described herein as follows: (i) isolating mitochondria; (ii) incubating mitochondria with [14C]malonate with and without a test compound; and (iii) determining the quantity of lipids and/or fatty acids produced. By comparing the amount of product in the presence of the test compound to the amount in its absence, the degree of inhibition can be determined. Also, since mmFAS does not make fat when incubated with acetyl-CoA, [14C]acetate may be added as a negative control.

Another method of measuring mmFAS activity is to measure FAS activity in cells from tissues that do not express Type I FAS, such as the heart and skeletal muscle tissue. Another method involves comparing a test compound to a compound that is a specific Type I FAS inhibitor. (See Rivkin et al., Bioorganic & Medicinal Chemistry Letters 16: 4620-4623, 2006). Another method involves the isolation of the mitochondrial matrix prior to measuring individual enzymes of the mitochondrial FAS complex.

B. Mitochondrial Membrane Depolarization

The methods described herein relate to screening compounds that are cytotoxic to tumor cells. One method of determining whether a compound is cytotoxic to tumor cells is to analyze mitochondrial membrane potential. This may be accomplished using kits known in the art. (See Cultek and Cayman Chemical websites). For example, one assay kit uses a fluorescent compound, JC-1, to signal the loss of mitochondrial membrane potential. In healthy non-apoptotic cells, the dye stains the mitochondria bright red. The negative charge established by the intact mitochondrial membrane potential allows the lipophilic dye, bearing a delocalized positive charge, to enter the mitochondrial matrix where it accumulates. When the critical concentration is exceeded, J-aggregates form which become fluorescent red. On the other hand, in apoptotic cells, the mitochondrial membrane potential collapses, and the JC-1 cannot accumulate within the mitochondria. In these cells, JC-1 remains in the cytoplasm in a green fluorescent monomeric form. Apoptotic cells, showing primarily green fluorescence, are easily differentiated from healthy cells which show red and green fluorescence.

The red JC-1 aggregates may be measured with a fluorimeter (Wallac 1420 Victor2, Perkin Elmer). To measure the red aggregate staining, the excitation wavelength is 550 nM and emission wavelength is 600 nM. Average values (600 nM emission) are plotted against concentrations of test compounds and the IC50 is determined using linear regression or if non-linear, using non-linear regression (Prism 4.0, Graph Pad Software).

C. Cytotoxicity Assays

Another method of determining whether a compound inhibits tumor cells is to perform a cytotoxicity assay. (See, e.g., Rothman, S., J Clin. Pathol. 39: 672-676, 1986). For example, to measure the cytotoxicity of a test compound against cancer cells, cells (e.g., SKOV-3 cells or OVCAR-3) are plated in 96-well plates. Following overnight culture, the compound is added to the cells at specified concentrations. After 72 hours of incubation, cells are incubated for 4 hours with the XTT reagent as per manufacturer's instructions (Cell Proliferation Kit II (XTT) Roche Diagnostics, New Jersey), and the amount of color produced by viable cells is measured spectrophotometrically. 1050 is calculated by linear regression, plotting the color produced in treated cells as percent of control (untreated) cells versus drug concentrations.

It will be appreciated that those test compounds having low IC₅₀ values are likely to be better inhibitors. Test compounds having low IC₅₀ values in more than one of the assays described herein are more likely to better inhibitors. It will be further be appreciated that hydrazides are not preferable inhibitors. Indeed, in some embodiments, the invention excludes hydrazides. In particular, hydrazide-containing compound C93 (depicted below) is not contemplated for in any of the methods described herein.

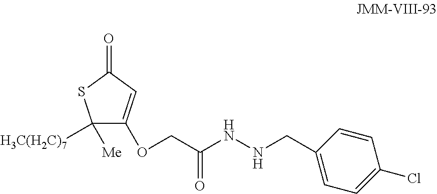

Preferably, the efficacy of compounds shown to be effective inhibitors in any of the above assays are confirmed by testing the reduction of tumor size in vivo using xenograft model systems.

Treatment Based on Inhibition of Type II Fatty Acid Synthesis

The invention provides a method for ameliorating tumor burden in mammals having a malignancy, particularly a tumor, more particularly a carcinoma. Malignancy may be reduced in such mammals by administering to the mammal one or more inhibitors that interfere with fatty acid synthesis or utilization via mmFAS. These inhibitors are cytotoxic to tumor cells which express mmFAS, and administration which results in reduction of Type II fatty acid synthesis and utilization by the tissue and/or reduction of mmFAS activity in biological fluids of these mammals will reduce tumor burden.

A. Susceptible Patients

Characteristic carcinomas amenable to treatment according to this invention include those of bladder, salivary gland, skin adnexae, bile duct, endocervix, ectocervix, and vagina, esophagus, nasopharynx and oropharynx, or those of germ cell origin, and mesothelioma. In particular, carcinomas or adenocarcinomas of the stomach, endometrium, kidney, liver and lung, as well as melanoma are treatable according to this invention. Breast, colon and rectum, prostate, and ovary, are especially suitable types of adenocarcinomas for the application of this therapy. In addition, hematopoietic malignancies including multiple myeloma and leukemia are also treatable according to this invention. The methods of this invention may be used to treat mammals suffering from similar malignancies.

The methods of this invention contemplate treatment of tumors having cells that express mmFAS or depend on endogenous fatty acid (synthesized within the mitochondria). Preferably, methods of this invention will be applied to mammals with malignancies having cells which over-express mmFAS.

Tumor cell sensitivity to mammalian mitochondrial fatty acid synthesis inhibitors may vary continuously with mmFAS levels. Since many tumor cells are extremely dependent on endogenous mammalian mitocondrial fatty acid synthesis, lower mmFAS activity levels need not exclude a specific tumor as a candidate for therapy with mammalian mitochondrial fatty acid synthase inhibitors. The presence of mmFAS in cells of the carcinoma may be detected by any suitable method including activity assays or stains, immunoassays using anti-mmFAS antibodies, assays measuring mmFAS mRNA, and the like.

While it is preferred that the presence of mmFAS be determined prior to treatment, the skilled clinician will recognize that such determination is not always necessary. Treatment of a carcinoma patient with an inhibitor of Type II fatty acid synthesis, particularly a mmFAS inhibitor, which results in reduction of tumor burden will be understood to correlate with the presence of mmFAS in the tumor. Where a carcinoma patient can be successfully treated by the methods of this invention, independent determination of mmFAS may be unnecessary. Such empirical treatment of carcinomas of the type usually found to express mmFAS is also within the contemplation of this invention.

B. Inhibitors of Mammalian Mitochondrial Fatty Acid Synthesis

The mammal or tumor patient may be treated according to the methods of this invention by administering a mammalian mitochondrial fatty acid synthesis inhibitor to the patient. Inhibitors whose administration is within the contemplation of this invention may include any compound that shows demonstrable inhibition of the mammalian mitochondrial fatty acid synthase or mmFAS biosynthesis pathway. The mmFAS inhibitors contemplated herein may also inhibit Type I FAS. Alternatively, the inhibitors preferentially inhibit mmFAS over Type I FAS. In one embodiment, the test compound the IC₅₀ for inhibition of mmFAS is lower than its IC₅₀ inhibition of Type I FAS activity. In another embodiment, the ratio of IC₅₀ against Type I is at least ten, five, or two fold higher than the IC₅₀ for Type II. In another alternative, the inhibitors do not inhibit Type I FAS.

The present invention relates to a class of compounds that are useful to inhibit the enzyme activity of mmFAS, thus, inhibiting one or more of the enzymatic steps of fatty acid synthesis. As described herein, a wide variety of compounds have been shown to inhibit mmFAS, and selection of a suitable mmFAS inhibitor for treatment of carcinoma patients is within the skill of the ordinary worker in this art. MmFAS inhibitors can be identified by testing the ability of a compound to inhibit mammalian mitochondrial fatty acid synthase activity using purified enzyme. Fatty acid synthase activity can be measured radioactively by measuring the incorporation of radiolabeled malonate in isolated mitochondria. (See Brody, S., Eur. J. Biochem. 187: 431-437, 1990) Suitable FAS inhibitors may be selected, for example, from those exemplified below.

Preferred fatty acid synthesis inhibitors are described in International Application No. PCT/US09/45945, entitled “Novel Compounds, Pharmaceutical Compositions Containing Same, and Methods of Use for Same,” filed on Jun. 2, 2008, and is herein incorporated by reference in its entirety. These inhibitors are also described below.

As used herein, “an alkyl group” denotes both straight and branched carbon chains with one or more carbon atoms. Exemplary alkyl groups include, but are not limited to, methyl, ethyl, n-propyl, i-propyl, n-butyl, sec-butyl, t-butyl, pentyl, hexyl, and the like.

As used herein, “substituted alkyl” is an alkyl group, as defined above, wherein one or more hydrogens of the alkyl group are substituted with 1 or more substituent groups as otherwise defined herein.

As used herein, “haloalkyl” refers to an alkyl group, as defined above, wherein one or more hydrogens of the alkyl group are substituted with 1 or more halogen atoms.

As used herein, “an alkoxy group” refers to a group of the formula alkyl-O—, where alkyl is as defined herein.

As used herein, “substituted alkoxy” refers to a substituted alkyl-O— group wherein the alkyl group is substituted as defined above.

As used herein, “haloalkoxy” refers to an alkoxy group, as defined above, wherein one or more hydrogens of the alkyl group are substituted with 1 or more halogen atoms.

As used herein, “alkenyl” refers to a saturated alkyl group containing one or more carbon to carbon double bonds.

As used herein, “an aryl group” denotes a structure derived from an aromatic ring containing only carbon atoms. Examples include, but are not limited to a phenyl or benzyl radical and derivatives thereof.

As used herein, “arylalkyl” denotes an aryl group having one or more alkyl groups not at the point of attachment of the aryl group.

As used herein, “alkylaryl” denotes an aryl group having an alkyl group at the point of attachment.

As used herein, “heteroaryl” encompasses a monocyclic aromatic ring containing five or six ring atoms consisting of carbon and at least one non-carbon atom, which may be but is not limited to one or more of the following: nitrogen, oxygen, sulfur, phosphorus, boron, chlorine, bromine, or iodine.

As used herein, “heterocyclic” refers to a monovalent saturated or partially unsaturated cyclic non-aromatic carbon ring group which contains at least one heteroatom, in certain embodiments between 1 to 4 heteroatoms, which may be but is not limited to one or more of the following: nitrogen, oxygen, sulfur, phosphorus, boron, chlorine, bromine, or iodine. In further non-limiting embodiments, the hetercyclic ring may be comprised of between 1 and 10 carbon atoms.

As used herein, “cycloalkyl” refers to a monovalent or polycyclic saturated or partially unsaturated cyclic non-aromatic group containing all carbon atoms in the ring structure, which may be substituted with one or more substituent groups defined herein. In certain non-limiting embodiments the number of carbons comprising the cycloalkyl group may be between 3 and 7.

In one embodiment, the class compounds of the present invention may be represented by Formula I:

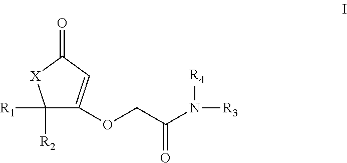

wherein X is comprised of a heteroatom which may be selected from any one of O, S, or NR, where R is H, alkyl, alkenyl, aryl, arylaklyl, or alkylaryl. R¹ and R² are independently selected from H, C₁-C₂₀ alkyl, cycloalkyl, alkenyl, aryl, arylalkyl, or alkylaryl. R³ and R⁴ are independently either a hydrogen atom or are members of a substituted or unsubstituted ring having 4-6 carbon atoms. In one embodiment, R³ and R⁴ are not both hydrogens. In another embodiment, if neither R³ and R⁴ is a hydrogen, then they together form an optionally substituted ring structure having 4-6 carbon atoms.

In further embodiments R³ is comprised of a hydrogen and R⁴ is comprised of a hydrogen, aryl group, a heteroaryl group, or a heterocyclic ring group having 4 to 6 carbon atoms wherein ring moiety of R⁴ is optionally substituted with one or more of a halogen atom, a C₁-C₃ alkyl group, a C₁-C₃ haloalkyl group, —OR⁵—SR⁵—CN, —CONH₂—SO₂NH₂, —C(O)OR⁶, —CONHR⁷ or a 5- or 6-membered cycloalkyl or heterocyclic ring. The latter 5- or 6-membered cycloalkyl or heterocyclic ring is optionally aromatic, optionally fused to two adjacent atoms of R⁴, and/or is optionally substituted with one or more R⁵ substitutent groups.

In an alternative embodiment, and as discussed in greater detail below, R³ and R⁴ together, along with the atoms and bonds to which they are attached, form a 5-7 membered heterocyclic ring having at least one nitrogen atom within the ring structure.

R⁵ is comprised of any one of a C₁-C₈ alkyl, C₁-C₈ alkoxy, aryl, alkylaryl, arylalkyl, which may be optionally substituted with one or more halogen atoms, C₁-C₃ alkyl groups, C₁-C₃ alkoxy groups, C₁-C₁ haloalkyl groups, or C₁-C₃ haloalkoxy groups.

R⁶ is comprised of a C₁-C₈ alkyl group. R⁷ is comprised of a C₁-C₈ alkyl, allyl group, a morpholine, a piperazine, an piperazine where the free nitrogen is substituted with R⁵, or a 5- or 6-membered heterocycle containing N, O, S or any combination thereof.

In another embodiment, the compounds of the present invention may be comprised of either an oxygen or sulfur in the X position defined in formula I. To this end, these embodiments may be defined by formula IIa and IIb below:

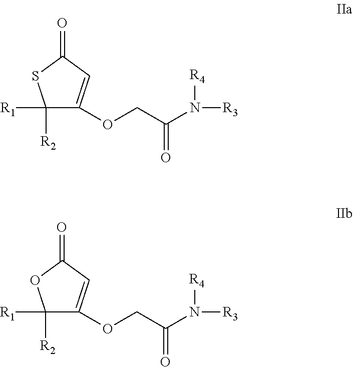

wherein each of R¹-R⁴ are defined within the embodiments discussed above.

In another embodiment, the compounds of the present invention are compounds of the formula I, where X is an NH group. To this end, this embodiment may be defined by formula IIc below:

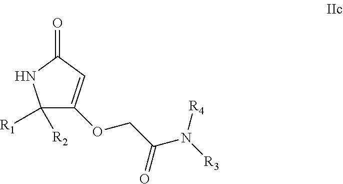

wherein each of R¹-R⁴ are defined within the embodiments discussed above.

In another embodiment, R³ is comprised of a hydrogen. R⁴ is comprised of an aryl group which may be optionally substituted with R⁸ and/or R^{8′} as set forth in formula III below:

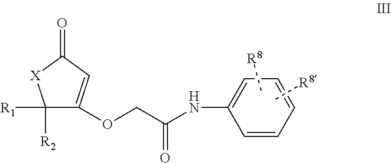

wherein each of R¹-R² are defined within the embodiments discussed above. R⁸ and R^{8′} are independently either absent from the structure or comprised of a halogen atom, a C₁-C₃ alkyl group, a C₁-C₃ haloalkyl group, —OR⁵—SR⁵—CN, —CONH₂—SO₂NH₂, —C(O)R⁶—CONHR⁷ or a 5- or 6-membered cycloalkyl or heterocyclic ring. The latter 5- or 6-membered cycloalkyl or heterocyclic ring is optionally aromatic, optionally fused to two adjacent carbon atoms of the aryl ring in the R⁴ position and/or is optionally substituted with R⁵, R⁵, R⁶, and R⁷ are any of the embodiments defined herein.

In a further embodiment of formula III, X may be comprised of an S or O as follows:

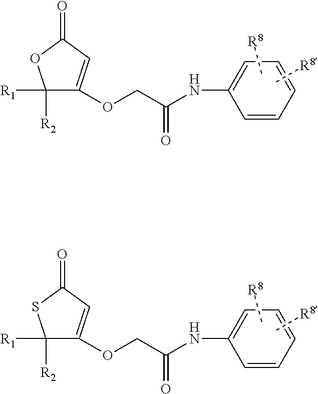

wherein R¹-R², R⁸ and R^{8′} are as defined herein.

In a further embodiment of formula III, X may be comprised of an S or O as follows:

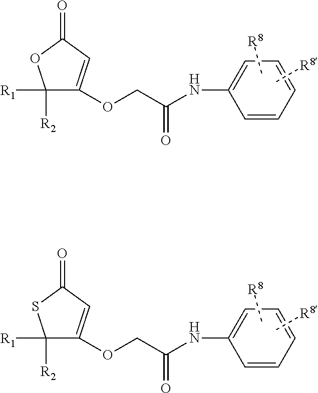

wherein R¹-R², R⁸ and R^{8′} are as defined herein.

In a further embodiment, R³ and R⁴ along with the atoms and bonds to which they are attached, form a 5-7 membered ring having at least one nitrogen atom within the ring structure. In certain embodiments the 5-7 membered ring may have at least two nitrogen atoms. In even further embodiments, R³ and R⁴ along with the atoms and bonds to which they are attached, form a 6-membered ring having two nitrogen atoms in a para position with respect to each other. In any of the foregoing embodiments the heterocyclic ring structure may be optionally substituted with R⁵ or any other substitution group discussed herein. To this end, embodiments of the foregoing may be represented by the structures of formula IV below:

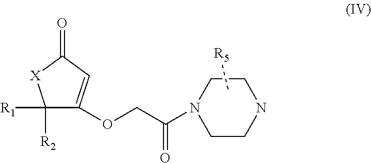

wherein R¹, R², and R⁵ are any of the embodiments defined above.

In a further embodiment of formula IV, X may be comprised of an S or O as follows:

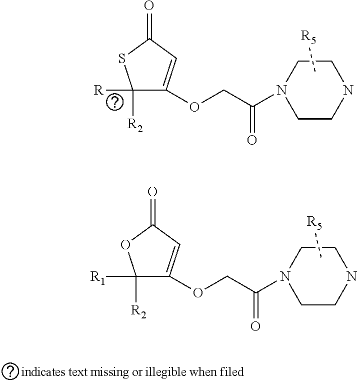

wherein R¹, R², and R⁵ are any of the embodiments defined above.

In certain non-limiting embodiments of the present invention R¹ is comprised of a straight or branched chain C₆-C₈ alkyl group. In further non-limiting embodiments, R¹ is comprised of a straight or branched chain C₈ alkyl group. In even further non-limiting embodiments, R¹ may be represented by the formula —(CH₂)₇CH₃.

In certain non-limiting embodiments of the present invention R² is comprised of a straight or branched chain C₁-C₃ alkyl group. In even further non-limiting embodiments, R² is comprised of a methyl group.

Based on the foregoing, the structures of formulas I, II, III, and IV may be of a compound having the following structure adapted as follows:

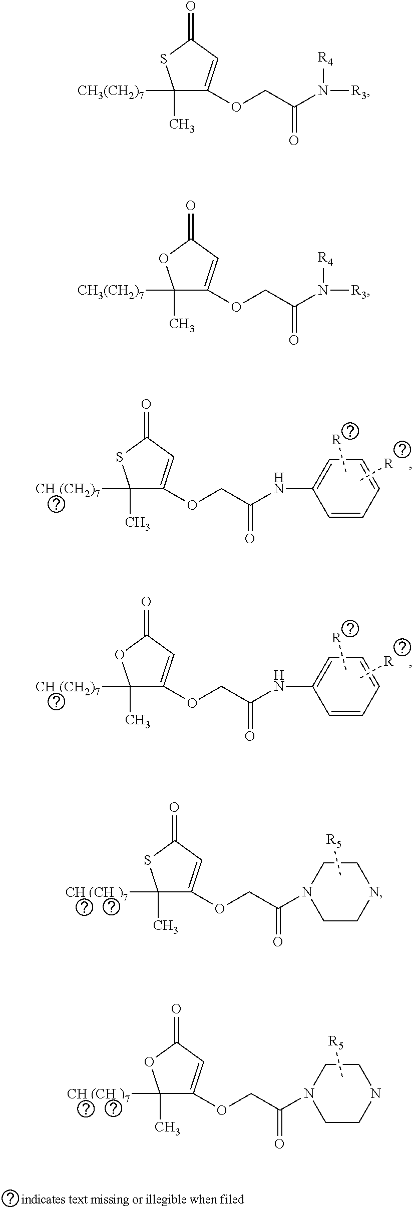

In certain embodiments the compound of the instant invention may be comprised of a compound having the following structure (referred to hereinafter as “C31”):

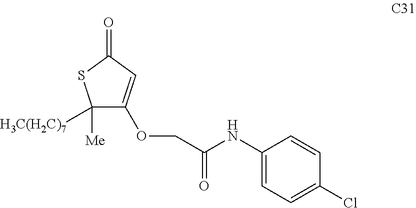

In certain embodiments the compound of the instant invention may be comprised of a compound having the following structure (referred to hereinafter as “C157”):

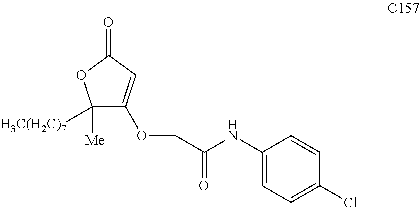

In certain embodiments the compound of the instant invention may be comprised of a compound having the following structure (referred to hereinafter as “C144”):

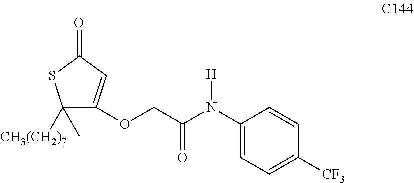

In certain embodiments the compounds of the instant invention may be comprised of a compound having the following structures (respectively referred to hereinafter as “C145”).

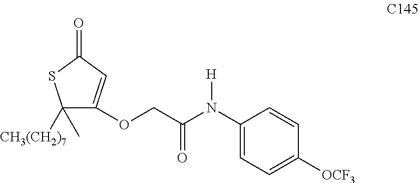

In certain embodiments the compounds of the instant invention may be comprised of a compound having the following structures (respectively referred to hereinafter as “C193”, “C138”, “C139”, “C141”, “C142”, “C178”, and “C181”).

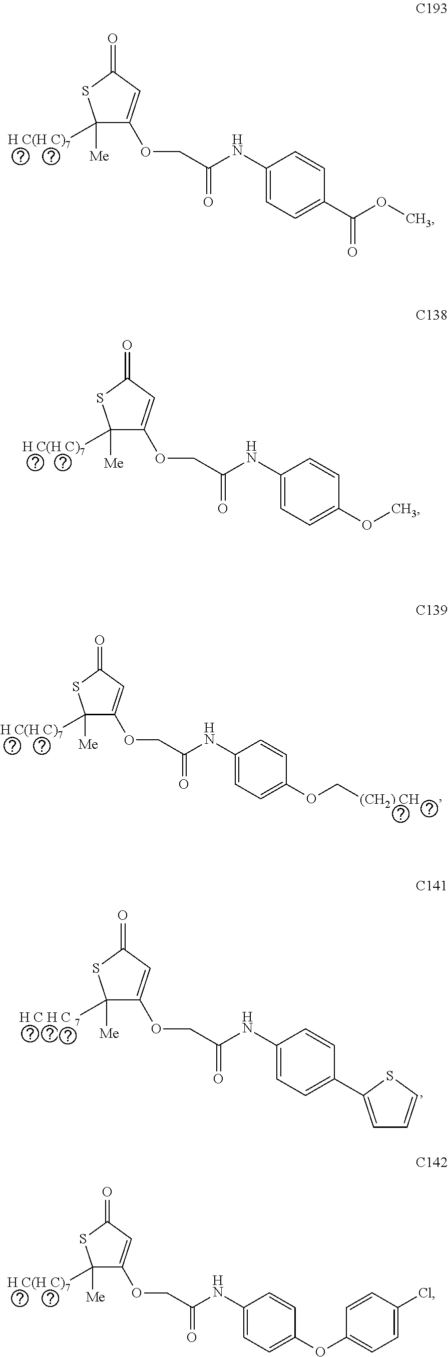

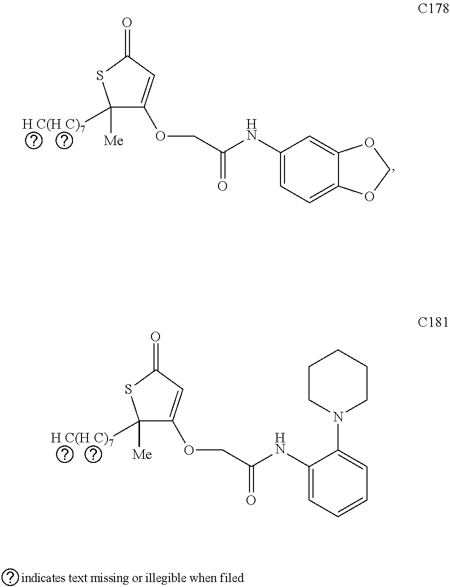

In certain embodiments the compounds of the instant invention may be any one of the following compounds:

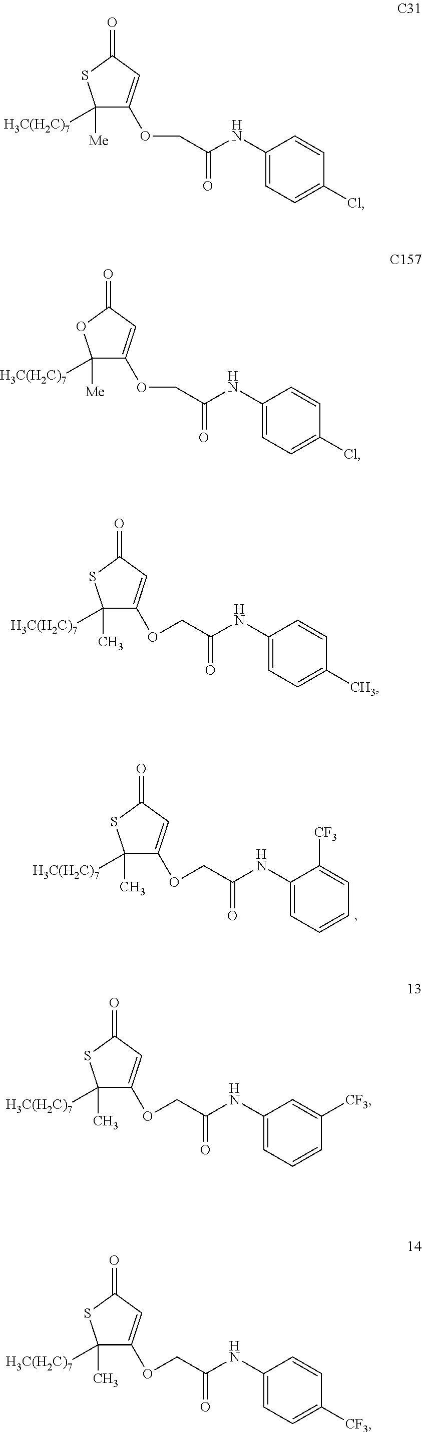

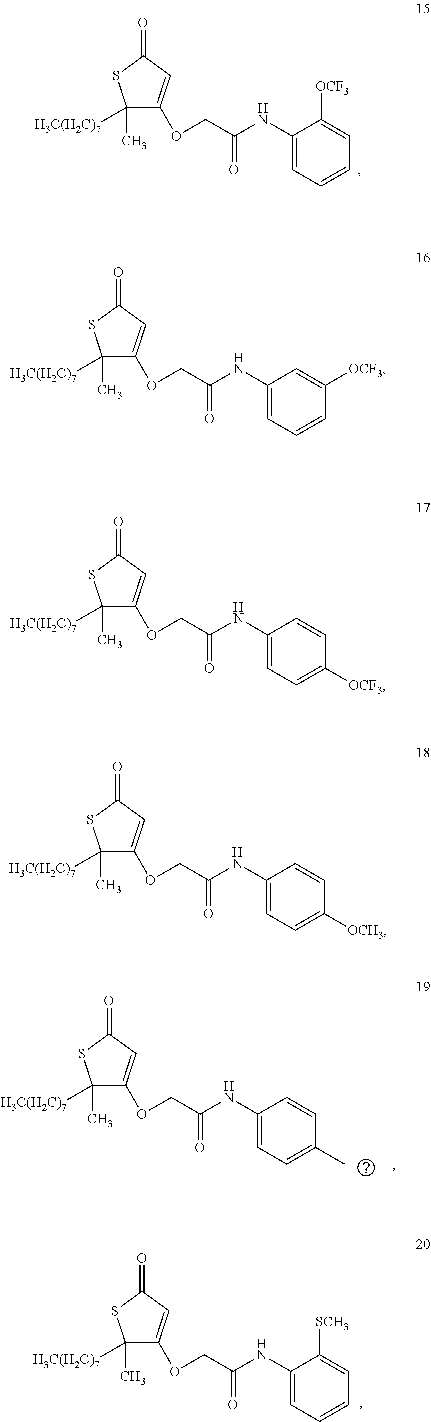

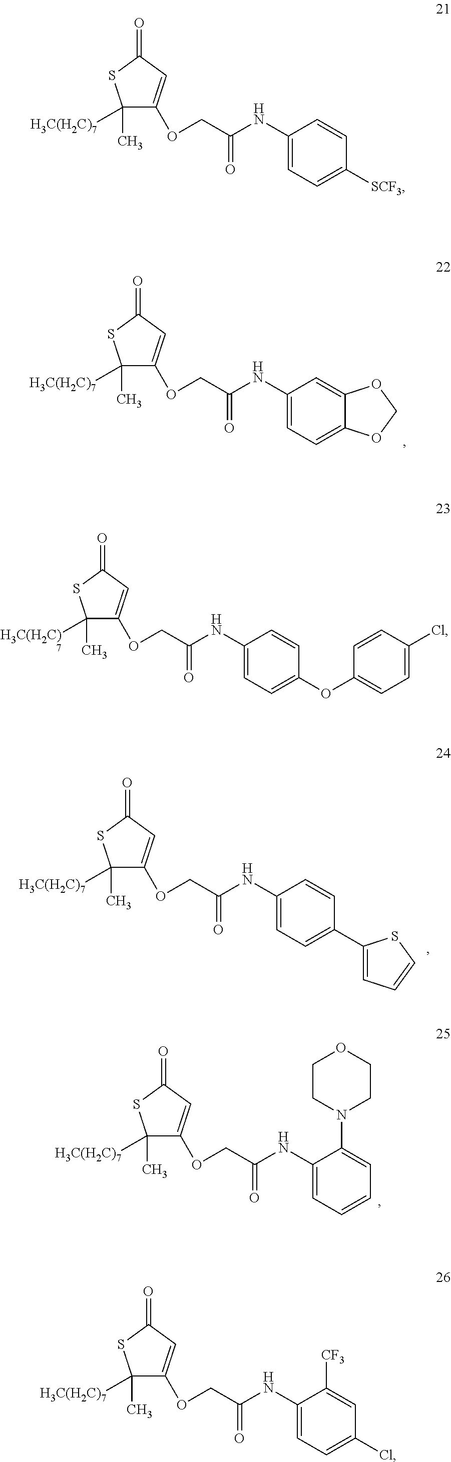

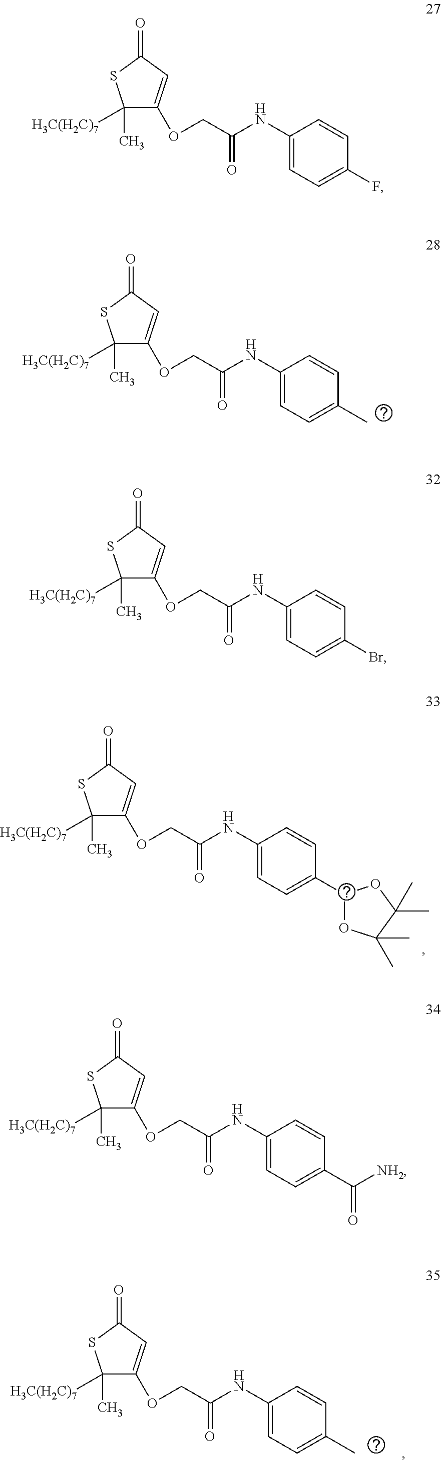

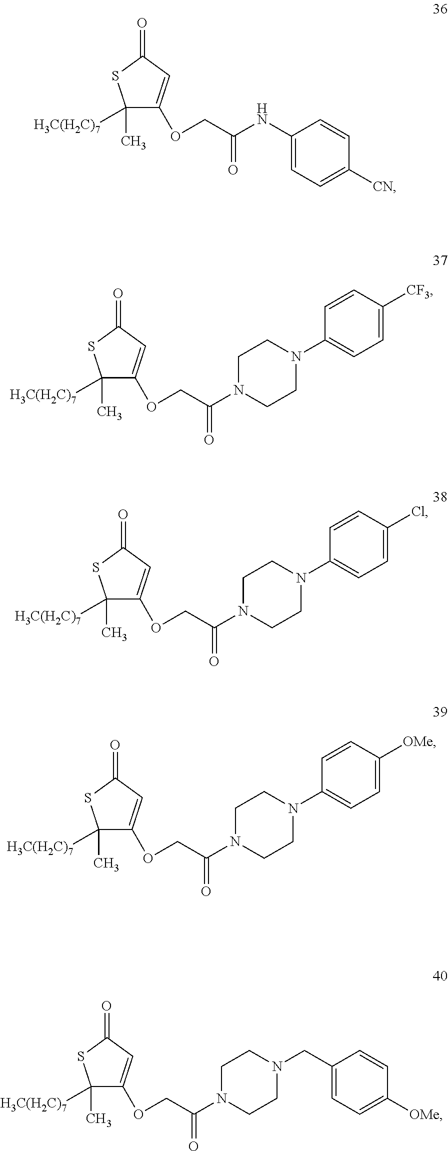

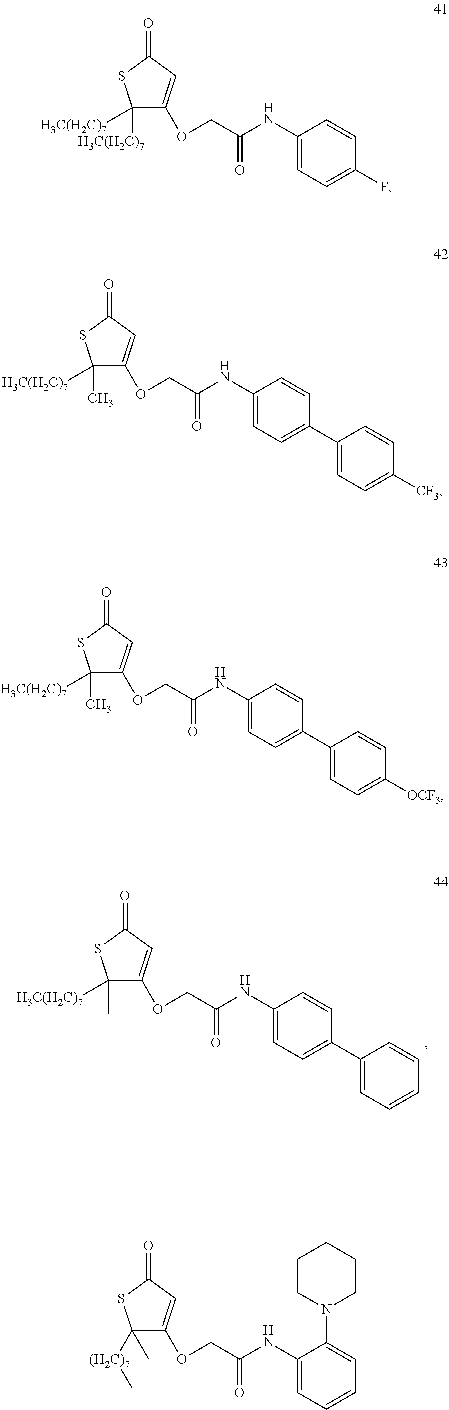

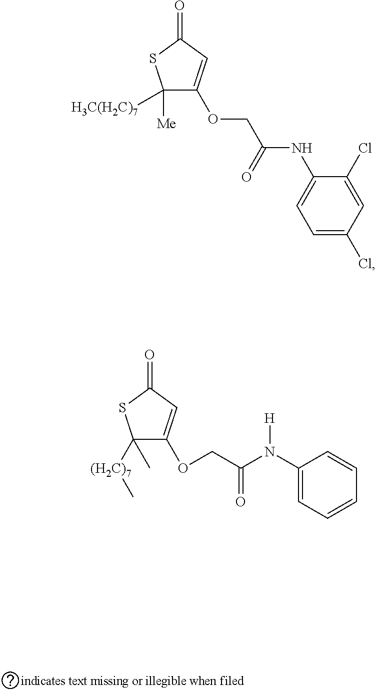

Compounds comprising hydrazides are potentially toxic and are less preferred or not useful in the methods described herein. In particular, compound 93 (depicted below) is not intended for therapeutic use.

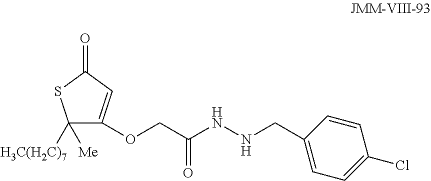

In certain embodiments, the mmFAS inhibitory compounds of the instant invention may be the diphenyl ether compounds disclosed in Published U.S. Application Number 2006/0041025, which is incorporated by reference as if fully set forth herein. Such compounds include the following compounds:
5-ethyl-2-phenoxyphenol;
2-phenoxy-5-propylphenol;
5-isopropyl-2-phenoxyphenol;
5-butyl-2-phenoxyphenol;
2-phenoxy-5-(t-butyl)phenol;
5-pentyl-2-phenoxyphenol;
5-hexyl-2-phenoxyphenol;
5-octyl-2-phenoxyphenol;
5-decyl-2-phenoxyphenol;
5-dodecyl-2-phenoxyphenol;
5-hexadecyl-2-phenoxyphenol;
2-phenoxy-5-tetracosylphenol;
5-butyl-2-(2-methylphenoxy)phenol;
5-butyl-2-(2-ethylphenoxy)phenol;
2-(2,6-dimethylphenoxy)-5-butyl-phenol;
2-(2-chlorophenoxy)-5-pentyl-phenol;
2-(4-nitrophenoxy)-5-pentyl-phenol;
2-(4-hydroxyphenoxy)-5-pentyl-phenol;
2-(4-methylphenoxy)-5-pentyl-phenol;
2-(4-chlorophenoxy)-5-pentyl-phenol;
3-ethyl-2-phenoxyphenol;
3-hexyl-2-phenoxyphenol;
3-octyl-2-phenoxyphenol;
3-dodecyl-2-phenoxyphenol;
3,5-dimethyl-2-phenoxyphenol;
5-ethyl-3-hexyl-2-phenoxyphenol;
3-hexyl-2-(2-fluorophenoxy)phenol;
3-decyl-2-(2-trifluoromethylphenoxy)phenol;
6-ethyl-2-hydroxyl-3-phenoxypyridine;
3-hydroxy-5-octyl-2-phenoxypyridine;
3-hydroxy-5-octyl-4-phenoxypyridine;
2-hydroxy-6-octyl-3-phenoxypyrazine;
5-octyl-2-(pyridin-4-yloxy)phenol;
2-hydroxy-6-octyl-3-(pyridin-4-yloxy)pyridine;
2-hydroxy-6-octyl-3-(pyridin-4-yloxy)pyrazine;
5-octyl-2-(pyrimidin-4-yloxy)phenol;
5-octyl-2-([1,3,5]triazin-2-yloxy)-phenol;
3-hydroxy-5-octyl-2-(pyrimidin-4-yloxy)pyridine;
3-hydroxy-5-octyl-2-(2-methylpyrimidin-4-yloxy)pyridine;
3-hydroxy-5-octyl-4-(2-methylpyrimidin-4-yloxy)pyridine;
6-ethyl-4-hydroxy-3-phenoxypyridine;
2-ethyl-4-hydroxy-5-phenoxypyrimidine; and
2,6-dimethyl-4-hydroxy-5-phenoxypyrimidine.
   In a particular embodiment, the mmFAS inhibitor is triclosan.

Unless otherwise specified, a reference to a particular compound of the present invention includes all isomeric forms of the compound, to include all diastereomers, tautomers, enantiomers, racemic and/or other mixtures thereof. Unless otherwise specified, a reference to a particular compound also includes ionic, salt, solvate (e.g., hydrate), protected forms, and prodrugs thereof. To this end, it may be convenient or desirable to prepare, purify, and/or handle a corresponding salt of the active compound, for example, a pharmaceutically-acceptable salt. Examples of pharmaceutically acceptable salts are discussed in Berge et al., 1977, “Pharmaceutically Acceptable Salts,” J. Pharm. Sci., Vol. 66, pp. 1-19, the contents of which are incorporated herein by reference.

Any compound that inhibits Type II fatty acid synthesis may be used to inhibit tumor cell growth, but of course, compounds administered to a patient must not be equally toxic to both malignant and normal (non-malignant) cells. Preferred inhibitors for use in the method of this invention are those with high therapeutic indices (therapeutic index is the ratio of the concentration which affects normal cells to the concentration which affects tumor cells). Inhibitors with high therapeutic index can be identified by comparing the effect of the inhibitor on two cell lines, one non-malignant line, such as normal hepatocytes, and one carcinoma line which has been shown to express high levels of mmFAS. Cells with the preferred level of mammalian mitochondrial fatty acid synthesis activity are easily obtained by the skilled worker, and examples of publicly available cell lines discussed in the Examples.

Inhibitors can be characterized by the concentration required to inhibit cell growth by 50% or to induce apoptosis in 50% of the tumor cells (IC₅₀ or LD₅₀). MmFAS inhibitors with high therapeutic index will, for example, be growth inhibitory to the carcinoma cells at a lower concentration (as measured by IC₅₀) than the IC₅₀ for the non-malignant cells. Inhibitors whose effects on these two cell types show greater differences are more preferred.

Mammalian mitochondrial fatty acid synthesis inhibitors are also useful in conjunction with other chemotherapeutic agents. Since no presently prescribed cancer chemotherapeutic agents are specifically active against the mammalian mitochondrial fatty acid synthase pathway, mmFAS inhibitors will complement existing anti-cancer drugs, particularly antimetabolic drugs that target other anabolic or catabolic pathways. (For protocol, see Menendez, J., Int. J. Cancer 115: 9-35, 2005).

Inhibitors of Type II fatty acid synthesis may be expected to be particularly effective in combination with chemotherapeutic agents that target rapidly cycling cells. Alternatively, Type II fatty acid synthesis inhibitors may be administered to supplement a chemotherapeutic regime based on antineoplastic agents known to be effective against the particular tumor type being treated. In particular, use of Type II fatty acid synthesis inhibitors to prevent the growth of a small proportion of undetected but highly virulent cells in conjunction with a therapeutic program using other agents is within the contemplation of this invention.

When tumors are treated by administration of a synergistic combination of at least one inhibitor of mammalian mitochondrial fatty acid synthesis and at least one inhibitor of either the enzymes which supply substrates to the fatty acid synthesis pathway or the enzymes that catalyze downstream processing and/or utilization of fatty acids, the therapeutic index will be sensitive to the concentrations of the component inhibitors of the combination. Optimization of the concentrations of the individual components by comparison of the effects of particular mixtures on non-malignant and mmFAS-expressing cells is a routine matter for the skilled artisan. The dose of individual components needed to achieve the therapeutic effect can then be determined by standard pharmaceutical methods, taking into account the pharmacology of the individual components.

The inhibitor of mammalian mitochondrial fatty acid synthesis, or the synergistic combination of inhibitors will be administered at a level (based on dose and duration of therapy) below the level that would kill the animal being treated. Preferably, administration will be at a level that will not irreversibly injure vital organs, or will not lead to a permanent reduction in liver function, kidney function, cardiopulmonary function, gastrointestinal function, genitourinary function, integumentary function, musculoskeletal function, or neurologic function. On the other hand, administration of inhibitors at a level that kills some cells which will subsequently be regenerated (e.g., endometrial cells) is not necessarily excluded.

C. Administration of Mammalian Mitochondrial Fatty Acid Synthesis Inhibitors

Inhibitors of mmFAS are preferably formulated in pharmaceutical compositions containing the inhibitor and a pharmaceutically acceptable carrier. The pharmaceutical composition may contain other components so long as the other components do not reduce the effectiveness of the FAS inhibitor so much that the therapy is negated. Suitable components include any of a multitude of possible additives commonly used in the art, such as carriers, excipients, diluting agents, fillers, or combinations thereof. Examples of these additives are water, alcohols, gelatin, saccharose, pectin, magnesium stearate, stearic acid, various oils of animal or plant origin, calcium hydrogen phosphate, mannitol, polyethylene glycol, or sugar alcohols. Pharmaceutically acceptable carriers are well known, and one skilled in the pharmaceutical art can easily select carriers suitable for particular routes of administration (Remington's Pharmaceutical Sciences, Mack Publishing Co., Easton, Pa., 1985).

Preferred pharmaceutical compositions include oral compositions, such as for example solid forms or liquid forms, rectal compositions such as suppositories, and parenteral compositions suitable for injection or infusion intravenously. The pharmaceutical compositions containing any of the inhibitors of this invention may be administered by parenteral (subcutaneously, intramuscularly, intravenously, intraperitoneally, intrapleurally, intravesicularly or intrathecally), topical, oral, rectal, or nasal route, as necessitated by choice of drug, tumor type, and tumor location.

Dose and duration of therapy will depend on a variety of factors, including the therapeutic index of the drugs, tumor type, patient age, patient weight, and tolerance of toxicity. Dose will generally be chosen to achieve serum concentrations from about 0.001 μg/ml to about 100 μg/ml, preferably about 0.020 μg/ml to about 20 μg/ml. Preferably, initial dose levels will be selected based on their ability to achieve ambient concentrations shown to be effective in in vitro models, such as that used to determine therapeutic index, and in vivo models and in clinical trials, up to maximum tolerated levels. It is understood that the effective amount will normally be determined by a prescribing physician and that this amount will vary according to metabolic parameters and the individual response profile of the patient, such as the severity of the subjects symptoms and the potency of specific compound being considered for demonstration. Standard procedure in oncology requires that chemotherapy be tailored to the individual patient and the circulatory concentration of the chemotherapeutic agent be monitored regularly. The dose of a particular drug and duration of therapy for a particular patient can be determined by the skilled clinician using standard pharmacological approaches in view of the above factors. The response to treatment may be monitored by monitoring tumor burden in the patient. The skilled clinician will adjust the dose and duration of therapy based on the response to treatment revealed by these measurements.

D. Suppression of Mammalian Mitochondrial FAS Biosynthesis

While the mmFAS inhibitors discussed herein are typically small molecule compounds that directly inhibit the enzyme, it will be readily apparent to the skilled clinician that specific prevention of mmFAS biosynthesis is an equivalent procedure which will accomplish the same desired result. This may be accomplished by selectively degrading mRNA encoding one of the mmFAS polypeptides or otherwise interfering with its transcription and/or translation. This may be accomplished, for instance, by introduction of a ribozyme specific for one of the mmFAS mRNA, antisense RNA complementary to the nucleic acid sequence of one of the mmFAS, or inhibition of one of the mmFAS via siRNA, microRNA, or RNAi technology. (See, e.g., Oh, Y. and Park, T., Advanced Drug Delivery Reviews 61: 850-862, 2009; Croce, C., Nature 10: 704-714, 2009; Garzon et al., Annu. Rev. Med. 60:167-179, 2009; Quon, K. and Kassner, P., Expert Opin. Ther. Targets 13(9): 1027-1035, 2009; Feng et al., J Biol Chem 284:11436-45, 2009).

The sequences of human mmFAS are known in the art:
phosphopantetheine transferase (See Joshi et al., J. Bio. Chem. 278(35): 33142-33149, 2003)
malonyl transferase and acyl carrier protein (See Zhang et al., J. Bio. Chem. 278(41): 40067-40074, 2003)
ketoacyl synthase (See Zhang et al., J. Bio. Chem. 280(13): 12422-12429, 2005)
3 hydroxyacyl-thioester dehydratase (See Autio et al., FASEB J., 22: 569-578, 2008)
enoyl thioester reductase (ETR) (See Miinalainen et al., J. Bio. Chem., 278(22): 20154-20161, 2003)
3-ketoacyl reductase (See Chen et al., FASEB J., 23: 3682-3691, 2009)

Antisense therapy, for example, involves an expression vector containing at least a portion of the sequence encoding human one of the mmFAS polypeptides operably linked to a promoter such that it will be expressed in antisense orientation. As a result, RNA which is complementary to and capable of binding or hybridizing to mmFAS mRNA will be produced. Upon binding to mRNA for mmFAS, translation of that mRNA is prevented, and consequently mmFAS is not produced. Production and use of antisense expression vectors is described in more detail in U.S. Pat. No. 5,107,065 and U.S. Pat. No. 5,190,931, both of which are incorporated herein by reference.

The expression vector material is generally produced by culture of recombinant or transfected cells and formulated in a pharmacologically acceptable solution or suspension, which is usually a physiologically-compatible aqueous solution, or in coated tablets, tablets, capsules, suppositories, inhalation aerosols, or ampules, as described in the art, for example in U.S. Pat. No. 4,446,128, incorporated herein by reference. The vector-containing composition is administered to a mammal in an amount sufficient to transfect a substantial portion of the target cells of the mammal. Administration may be any suitable route, including oral, rectal, intranasal or by intravesicular (e.g. bladder) instillation or injection where injection may be, for example, transdermal, subcutaneous, intramuscular or intravenous. Preferably, the expression vector is administered to the mammal so that the tumor cells of the mammal are preferentially transfected. Determination of the amount to be administered will involve consideration of infectivity of the vector, transfection efficiency in vitro, immune response of the patient, etc. A typical initial dose for administration would be 10-1000 micrograms when administered intravenously, intramuscularly, subcutaneously, intravesicularly, or in inhalation aerosol, 100 to 1000 micrograms by mouth, or 105 to 1010 plaque forming units of a recombinant vector, although this amount may be adjusted by a clinician doing the administration as commonly occurs in the administration of other pharmacological agents. A single administration may usually be sufficient to produce a therapeutic effect, but multiple administrations may be necessary to assure continued response over a substantial period of time.

Further description of suitable methods of formulation and administration according to this invention may be found in U.S. Pat. Nos. 4,592,002 and 4,920,209, incorporated herein by reference.

E. Non-Invasive Therapy Using Inhibitors of Fatty Acid Synthesis

In a preferred mode, the inhibitor of fatty acid synthesis is formulated in a pharmaceutical composition and applied to an externally accessible surface of a patient having a neoplastic lesion in an externally accessible surface. Externally accessible surfaces include all surfaces that may be reached by non-invasive means (without cutting or puncturing the skin), including the skin surface itself, mucus membranes, such as those covering nasal, oral, gastrointestinal, or urogenital surfaces, and pulmonary surfaces, such as the alveolar sacs.

Cancers likely to benefit from topical therapy directed at endogenous mammalian mitochondrial fatty acid synthesis basal cell carcinoma squamous cell carcinoma, include but not limited to the following subtypes: actinic keratosis actinic cheilitis, cornu cutaneum, keratoacanthoma, squamous cell carcinoma in situ, dysplasia; apocrine carcinoma; eccrine carcinoma; sebaceous carcinoma; merkel cell tumor; Paget's disease (a cutaneous form of breast cancer); Extramammary Paget's disease; cutaneous melanoma; transitional cell carcinoma; and other in situ or dysplastic lesions.

Neoplastic lesions in externally accessible surfaces are preferably treated by non-invasive administration of an inhibitor of fatty acid synthesis or by local invasive administration, such as intra-lesional injection, where the administration is substantially non-systemic. Administration of a pharmaceutical composition containing a fatty acid synthesis inhibitor is substantially non-systemic where biological effects of the inhibitor can be observed locally, but the systemic concentration of the inhibitor is below the level required for therapeutic effectiveness and also below the level at which the inhibitor would generate adverse side effects. Non-invasive administration includes (1) topical application to the skin in a formulation, such as an ointment or cream, which will retain the inhibitor in a localized area; (2) direct topical application to oropharyngeal tissues; (3) oral administration of non-absorbable agents or agents that are inactivated upon absorption; (4) nasal administration as an aerosol; (5) intravaginal application of the inhibitor formulated in a suppository, cream or foam; (6) direct application to the uterine cervix; (7) rectal administration via suppository, irrigation or other suitable means; (8) bladder irrigation; and (9) administration of aerosolized formulation of the inhibitor to the lung. Aerosolization may be accomplished by well known means, such as the means described in International Patent Publication WO 93/12756, pages 30-32, incorporated herein by reference.

While inhibitors of fatty acid synthesis formulated as described previously may be used for non-systemic administration, a preferred strategy is to administer these compounds locally or topically in gels, ointments, solutions, impregnated bandages, liposomes, or biodegradable microcapsules. Compositions or dosage forms for topical application may include solutions, lotions, ointments, creams, gels, suppositories, sprays, aerosols, suspensions, dusting powder, impregnated bandages and dressings, liposomes, biodegradable polymers, and artificial skin. Typical pharmaceutical carriers which make up the foregoing compositions include alginates, carboxymethylcellulose, methylcellulose, agarose, pectins, gelatins, collagen, vegetable oils, mineral oils, stearic acid, stearyl alcohol, petrolatum, polyethylene glycol, polysorbate, polylactate, polyglycolate, polyanhydrides, phospholipids, polyvinylpyrrolidone, and the like.

A particularly preferred formulation for fatty acid synthesis inhibitors is in liposomes. Liposomes containing fatty acid synthesis inhibitors according to this invention may be prepared by any of the methods known in the art for preparation of liposomes containing small molecule inclusions. Liposomes that are particularly suited for aerosol application to the lungs are described in International Patent Publication WO 93/12756, pages 25-29, incorporated The concentrations of the active agent in pharmaceutically acceptable carriers may range from 1 pM to 100 mM. The dose used in a particular formulation or application will be determined by the requirements of the particular type of infection and the constraints imposed by the characteristics and capacities of the carrier materials. Dose and duration of therapy will depend on a variety of factors, including the therapeutic index of the drugs, type of infection, patient age, patient weight, and tolerance of toxicity. Preferably, initial dose levels will be selected based on their ability to achieve ambient concentrations shown to be effective against the target organism in-vitro, such as the model system used to determine therapeutic index, and in vivo models and in clinical trials, up to is maximum tolerated levels. The dose of a particular drug and duration of therapy for a particular patient can be determined by the skilled clinician using standard pharmacological approaches in view of the above factors. The response to treatment may be monitored by monitoring tumor burden in the patient. The skilled clinician will adjust the dose and duration of therapy based on the response to treatment revealed by these measurements.

### Examples

The following examples are included for illustrative purposes, and are not intended to limit the scope of the invention, which is limited only by the appended claims.

### Example 1

### Inhibition of Fatty Acid Synthesis

This example explores the effect of representative compounds on various fatty acid synthetic measures: (i) activity of the isolated fatty acid synthase multidomain enzyme, (ii) incorporation of labeled carbon from acetate into the lipid fraction of whole cells, and (iii) incorporation of labeled carbon from malonate into the lipid fraction of isolated mitochondria. In addition to various test compounds described above, a control compound was included in these studies based on activity against Type I human FAS reported in Rivkin, et al. (2006), “3-Aryl-4-hydroxyquinolin-2(1H)-one derivatives as type I fatty acid synthase inhibitors,” Bioorg Med Chem Lett 16, 4620-4623 (see Merck Research Laboratory compound 7d in tables 2 and 3, designated “Riv7d” hereinafter).

### (i) Activity of the Isolated Fatty Acid Syntase Multidomain Enzyme

Measurement of FAS Activity Using Purified Human Type I FAS

Human Type I FAS was purified from cultured ZR-75-1 human breast cancer cells obtained from the American Type Culture Collection. The procedure, adapted from Linn et al., 1981, and Kuhajda et al., 1994, utilizes hypotonic lysis, successive polyethyleneglycol (PEG) precipitations, and anion exchange chromatography. ZR-75-1 cells are cultured at 37° C. with 5% CO₂ in RPMI culture medium with 10% fetal bovine serum, penicillin and streptomycin.

Ten T150 flasks of confluent cells are lysed with 1.5 ml lysis buffer (20 mM Tris-HCl, pH 7.5, 1 mM EDTA, 0.1 mM phenylmethanesulfonyl fluoride (PMSF), 0.1% Igepal CA-630) and bounce homogenized on ice for 20 strokes. The lysate is centrifuged in a JA-20 rotor (Beckman) at 20,000 rpm for 30 minutes at 4° C. and the supernatant is brought to 42 ml with lysis buffer. A solution of 50% PEG 8000 in lysis buffer is added slowly to the supernatant to a final concentration of 7.5%. After rocking for 60 minutes at 4° C., the solution is centrifuged in a JA-20 rotor (Beckman) at 15,000 rpm for 30 minutes at 4° C. Solid PEG 8000 is then added to the supernatant to a final concentration of 15%. After the rocking and centrifugation is repeated as above, the pellet is resuspended overnight at 4° C. in 10 ml of Buffer A (20 mM K₂HPO₄, pH 7.4). After 0.45 μM filtration, the protein solution is applied to a Mono Q 5/5 anion exchange column (Pharmacia). The column is washed for 15 minutes with buffer A at 1 ml/minute, and bound material is eluted with a linear 60-ml gradient over 60 minutes to 1 M KCl. Type I FAS typically elutes at 0.25 M KCl in three 0.5 ml fractions identified using 4-15% SDS-PAGE with Coomassie G250 stain (Bio-Rad). FAS protein concentration is determined using the Coomassie Plus Protein Assay Reagent (Pierce) according to manufacturer's specifications using BSA as a standard. This procedure results in substantially pure preparations of Type I FAS (>95%) as judged by Coomassie-stained gels.

Type I FAS activity is measured by monitoring the malonyl-CoA dependent oxidation of NADPH spectrophotometrically at OD₃₄₀ in 96-well plates (Dils et al and Arslanian et al, 1975). Each well contains 2 μg purified FAS, 100 mM K₂HPO₄, pH 6.5, 1 mM dithiothreitol (Sigma), and 187.5 mM P-NADPH (Sigma).

The assay is performed on a Molecular Devices SpectraMax Plus Spectrophotometer. The plate containing FAS, buffers, and controls is placed in the spectrophotometer heated to 37° C. Using the kinetic protocol, the wells are blanked on duplicate wells containing 100 μl of 100 mM K₂HPO₄, pH 6.5 and the plate is read at OD₃₄₀ at 10 sec intervals for 5 minutes to measure any malonyl-CoA independent oxidation of NADPH. The plate is removed from the spectrophotometer and malonyl-CoA (67.4 μM, final concentration per well) and acetyl-CoA (61.8 μM, final concentration per well) are added to each well except to the blanks. The plate is read again as above with the kinetic protocol to measure the malonyl-CoA dependent NADPH oxidation. The difference between the ΔOD₃₄₀ for the malonyl-CoA dependent and non-malonyl-CoA dependent NADPH oxidation is the specific FAS activity. Because of the purity of the FAS preparation, non-malonyl-CoA dependent NADPH oxidation is negligible.

When determining the inhibitory effect of a test compound, the test compound is added prior to placing the plate into the spectrophotometer for temperature equilibration. Stock solutions of inhibitors are prepared in DMSO at 2, 1, and 0.5 mg/ml resulting in final concentrations of 20, 10, and 5 μg/ml when 1 μl of stock is added per well. For each experiment, cerulenin (Sigma) is run as a positive control along with DMSO controls, inhibitors, and blanks (no FAS enzyme) all in duplicate.

The inhibitory effect of a test compound may be quantified by IC₅₀ (the concentration of compound yielding 50% inhibition of FAS). The IC₅₀ is determined by plotting the ΔOD₃₄₀ for each inhibitor concentration tested, performing linear regression and computing the best-fit line, r² values, and 95% confidence intervals. Graphs of ΔOD₃₄₀ versus time are plotted by the SOFTmax PRO software (Molecular Devices) for each compound concentration. Computation of linear regression, best-fit line, r², and 95% confidence intervals are calculated using Prism Version 3.0 (Graph Pad Software).

When inhibition of Type I cytosolic FAS activity by compound C31 was determined using [¹⁴C]malonyl-CoA incorporation into lipid in MCF7 human breast cancer cell lysates described above, IC₅₀ was found to be 17.8 μM. This may be compared to the IC₅₀ of 136 nM reported for human Type I FAS by Riv7d using another assay technique in Rivkin, et al. (2006), “3-Aryl-4-hydroxyquinolin-2(1H)-one derivatives as type I fatty acid synthase inhibitors,” Bioorg Med Chem Lett 16, 4620-4623.

Measurement of FAS Activity Using Purified Rat Type I FAS

Highly purified rat FAS was obtained from starved and induced Sprague-Dawley rat livers by slight modification of a published procedure.xx [Abraham, S.; Smith, S. Methods Enzymol. 1975, 35B, 65-73.]. Enzyme activity was assayed spectrophotometrically by measuring the decrease in extinction at 340 nm that accompanies the oxidation of NADPH during the overall reaction. The enzyme reaction mixture contained 100 mM KH2PO4 (pH 6.6), 1 mM EDTA, 1 mM dithiothreitol (DTT), 0.10 mM acetylCoA, 0.20 mM NADPH, ˜3 μg FAS and escalating concentrations of inhibitor to be tested were combined in a final volume of 1 mL and assayed at 37° C. for 5 min to monitor malonylCoA-independent oxidation of NADPH. FAS activity was then initiated by addition of 0.2 mM malonyl-CoA.

The results demonstrated that C31 inhibits purified rat liver FAS with an IC₅₀ of approximately 41 μM (17.2±2.9 μg/mL). In contrast, Riv7d inhibits purified rat liver FAS with an IC50 of 37 nM. Thus, Riv7d is more efficacious at inhibiting purified cytosolic FAS than C 31.

(ii) Incorporation of Labeled Carbon into the Lipid Fraction of Whole Cells

MCF7 human breast cancer cells (a cell line which overexpresses FAS) were cultured in DMEM containing 5 mM glucose medium with 10% fetal calf serum, 0.01 mg/ml insulin and 1% penicillin/streptomycin. For measurement of fatty acid synthesis cells were cultured in 24-well plates at 5×10⁴ per well and incubated overnight. Fatty acid synthesis was assayed by a 2 h pulse of [U¹⁴C]acetate, 1 μCi/well, followed by Folch extraction and scintillation counting. Test compounds were added with the addition of drugs as described. Controls were treated with DMSO alone.

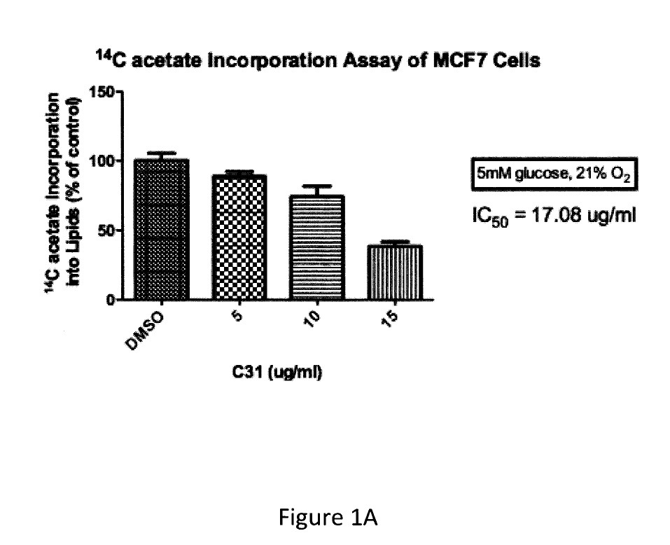

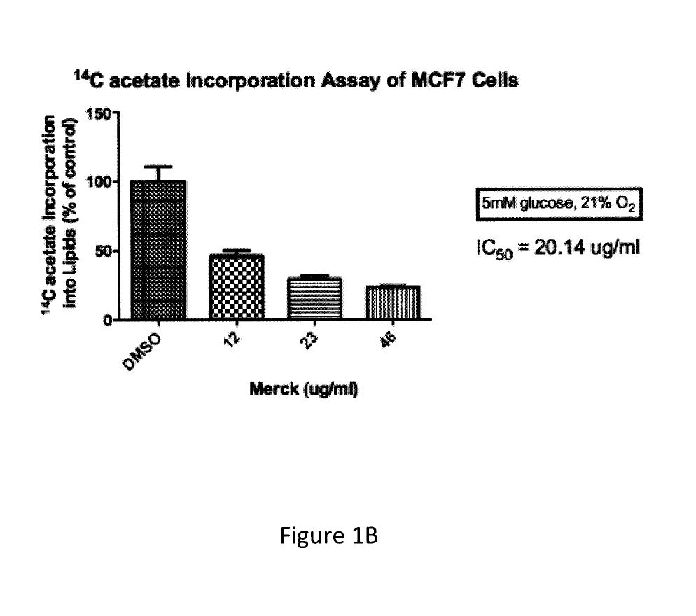

In the first experiment, we demonstrated using ¹⁴C-acetate incorporation into lipids that Riv7d and C31 were equally effective at inhibiting ¹⁴C-acetate incorporation into lipids in MCF7 cells, a human cancer cell line, in vitro. As shown in FIG. 1, Riv7d (1B) had an IC₅₀ of 20 μg/mL, while C31 (1A) displayed an IC₅₀ of 17 μg/mL. These data indicate that both Riv7d and C31 are effective at inhibiting FAS activity in intact cancer cells. In a second experiment using the same procedure, the IC₅₀ for other compounds was measured as shown in the table below. However, these assays do not discriminate between FAS I and FAS II activities.

| Compound | IC₅₀ |
|---|---|
| C31 | 13.6 μM |
| C144 | greater than 5.6 μM |
| C145 | 17.8 μM |

(iii) Incorporation of Labeled Carbon into the Lipid Fraction of Mitochondria

Mitochondria from MCF7 cells were prepared following manufacturer's protocol (Mitochondria Isolation Kit, Sigma, Saint Louis, Mo.). Rat liver mitochondria were isolated by homogenization with a tissue homogenizer in H-medium (220 mM mannitol, 70 mM sucrose, 2 mM HEPES, and 0.5 g/L of fatty acid free bovine serum albumin, pH 7.4). The homogenate was centrifuged at 1100 g for 3 min and washed twice with H-medium. The supernatants were pooled and centrifuged at 6800 g for 15 min. The pellet was then rinsed again and centrifuged at 3000 g for 3 min. The mitochondria supernatants were saved. The sediments were resuspended in H-medium and centrifuged at 3000 g for 3 min. The mitochondria supernatants were combined and centrifuged at 20,000 g for 15 min. The resulting mitochondria pellet were rinsed and centrifuged again at 20,000 g for 20 min.

Isolated mitochondria (100-200 μg) were mixed with 50 mM TES-KOH (pH 7.2), 10 mM MgCl2, 0.3 M sorbitol, 0.1% BSA, 1 mM each of ATP, ADP, NADH, NADPH, CoA, DTT and 1 μCi of [2-¹⁴C]malonic acid (50 mCi/mmol) in a total volume of 200 μl. The reaction was performed for 30 min at room temperature. C31, Riv7d or vehicle were added at the beginning of this reaction. At the end of 30 minutes, the mitochondria pellet was separated from supernatant by centrifugation at 14000 rpm for 5 min. After washing with 500 μl of 50 mM TES-KOH (pH 7.2) and 0.3 M sorbitol, the mitochondria were incubated in 200 μl of H₂O and 20 μl of 10 M KOH for 15 min at 65° C. Then the samples were added with 40 μl of 5 M H₂SO₄ and 20 μl of 1 mM palmitic acid (in 1 M acetic acid in isopropanol). The ^{14C} labeled fatty acids were twice extracted with hexane and sent to scintillation counting after drying.

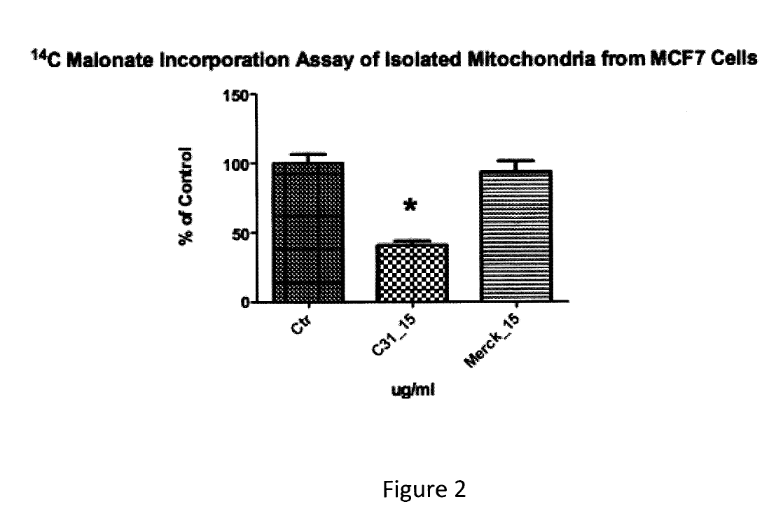

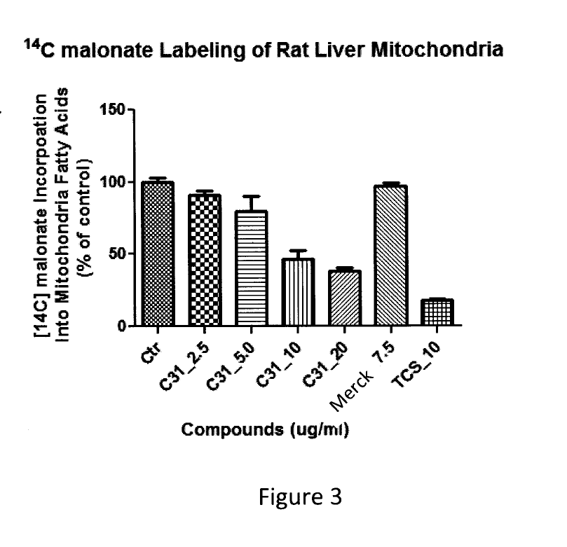

The results of these experiments show the ability of C31 or Riv7d to affect the incorporation of ¹⁴C-malonate into lipids of mitochondria isolated from MCF7 cells or rat liver—a test for fatty acid synthesis capacity. As shown in FIG. 2, compared to vehicle control, C31 inhibited the incorporation of malonate into lipids in a dose-dependent manner. In contrast, Riv7d was completely ineffective at inhibiting mitochondrial FAS activity. In a second set of experiments (FIG. 3), a dose-response curve was performed for C31 and its ability to inhibit ¹⁴C-malonate incorporation into mitochondrial fatty acids using mitochondria isolated from rat liver. C31 showed a dose-dependent inhibitory effect on mitochondrial fatty acid synthesis, whereas Riv7d had no effect. Triclosan (TCS) was used as a positive control, and showed an inhibitory effect on malonate incorporation. Triclosan is a known bacterial FAS inhibitor. In summary, C31 had an IC₅₀ of less than 4 μg per mL, Triclosan had an IC₅₀ of 3 μg per mL, whereas an IC₅₀ could not be determined for Riv7d due to its poor activity at inhibiting mitochondrial FAS.

### Example 2

### Effect on Energy Balance

This example explores the effect of representative compounds on various measures of cellular metabolism or energy balance: (i) cellular ATP level, (ii) AMP-activated protein kinase status, and (iii) polarization of mitochondrial membranes.

(i) Cellular ATP Level

As mitochondria serve as the energy production center of the cell, their main purpose is the generation of ATP. As a functional readout, cellular ATP levels may be measured to obtain an indication of mitochondrial status upon compound treatment. MCF-7 cells were cultured the standard medium, and ATP levels were measured by chemiluminescence.

MCF7 human breast cancer cells cultured in DMEM containing 5 mM glucose medium with 10% fetal calf serum, 0.01 mg/ml insulin and 1% penicillin/streptomycin. Cells were plated in 24-well cell culture plates at 2×10⁵ per well overnight. After drug treatment, cells were lysed using 500 μl of TE buffer (4 mM Tris, 0.25 mM EDTA, pH 7.4) and boiled at 95° C. for 7 min. ATP levels were measured within the linear range using the ATP BioLuminescence Kit CLSII (Roche) by following the manufacture's recommendation. Luminescence was detected using PerkinElmer Victor 1420.

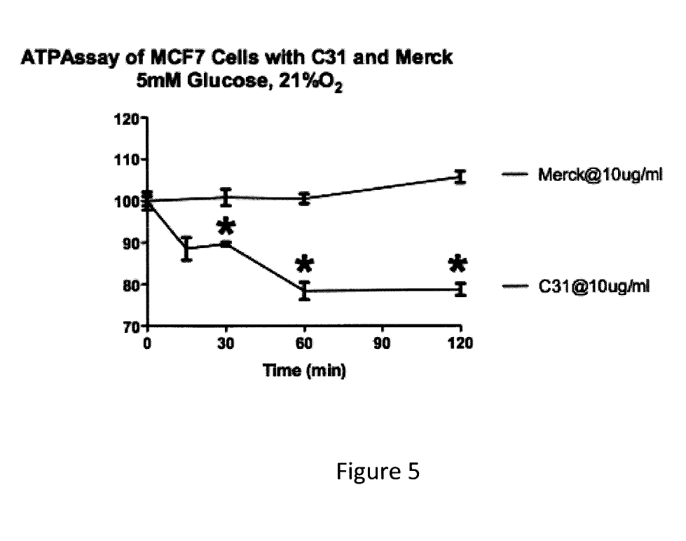

As shown in FIG. 5, ATP levels rapidly decreased following C31 treatment. Riv7d failed to induce any decrease in ATP levels. These data indicate that C31 rapidly and significantly compromises ATP levels in cancer cells, while Riv7d is not as effective. This result is consistent with the concept of this invention that mitochondrial FAS inhibition, not cytoplasmic FAS inhibition, is an effective target for cancer chemotherapy.

(ii) AMP-Activated Protein Kinase Status

Another indicator of cellular energy status and metabolic compromise is the activity on the cellular kinase AMP-activated protein kinase (AMPK). AMPK is a master energy sensor. AMPK regulates both enzyme activities and gene expression. When energy levels are low, and ATP levels fall with the concomitant rise in AMT levels, AMPK is activated and phosphorylated. When energy levels are surfeit, AMPK is inactivated and not phosphorylated. Thus, AMPK controls metabolic fluxes between anabolic and catabolic states.

We determined the ability of C31 or Riv7d to cause the activation of AMPK in a variety of cancer cells in vitro. SKOV3 cells were cultured in DMEM containing 5 mM glucose medium with 10% fetal calf serum, 0.01 mg/ml insulin and 1% penicillin/streptomycin. Antibodies to pAMPK and AMPK were obtained from Cell Signaling. Cells were grown in 6-well plates at 5×10⁴ per well and incubated overnight. Following drug treatment, the cells were harvested in Laemmli buffer and boiled for 5 minutes. Proteins were separated on a 4-15% SDS-PAGE gradient gel and transferred to a nitrocellulose membrane.

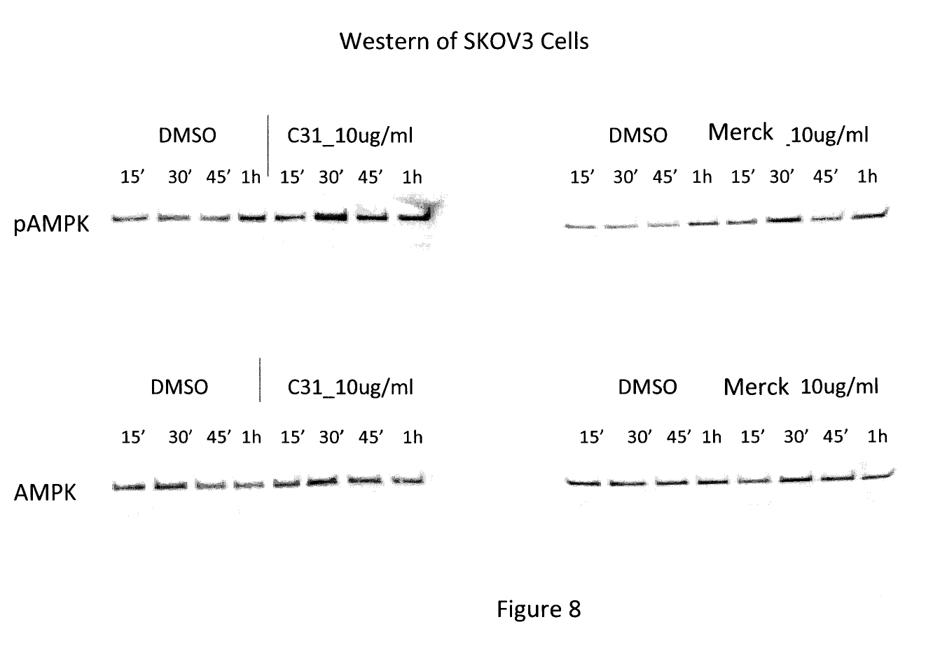

As shown by Western blot analysis (see FIG. 8), vehicle treatment did not affect pAMPK levels, a direct measure of AMPK activity. Riv7d was equally ineffective at activating pAMPK. In contrast, C31 caused a rapid activation of AMPK as demonstrated by the increased phosphorylation of AMPK on Western blot over time.

(iii) Depolarization of Mitochondrial Membranes

JC-1 staining is an indicator of mitochondrial membrane potential; with this assay, mitochondria within cells stained with this fluorescent dye emit a wavelength in the red spectrum when mitochondrial membrane potential is intact. When mitochondria are depolarized, the wavelength of the spectrum emitted changes to green. Therefore, the ratio of red/green fluorescence will give an indication of the intactness of the membrane potential of mitochondria, and thus mitochondrial function.

MCF7 cells were cultured in 24-well plates at 1×10⁴ per well and incubated overnight. Following drug inhibition the medium was removed and 1 μg/ml JC-1 (Molecular Probes) in DPBS was added to the cells. Plates were incubated for 30 min at 37° C., and washed two times with DPBS. Fluorescence from each well was measured at 530 nm/590 nm (excitation/emission) and by 485 nm/535 nm (excitation/emission) separately using PerkinElmer Victor 1420. The ratio of red to green fluorescence is a measure of mitochondrial membrane potential and thus mitochondrial function directly.

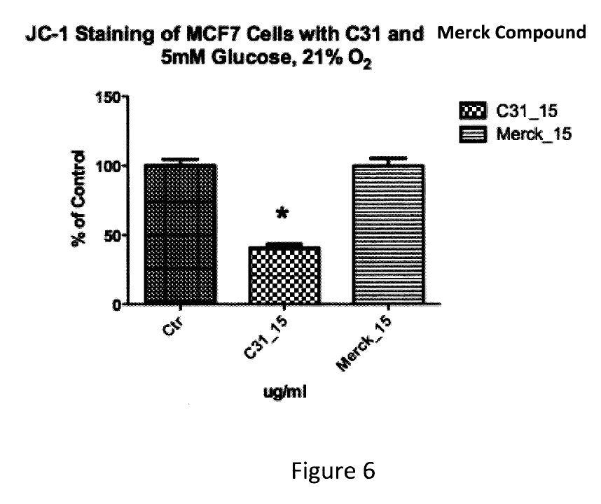

To evaluate mitochondrial integrity in whole cells, we performed JC-1 staining of cancer cells in vitro using C31, vehicle, or Riv7d. Compared to vehicle, C31 demonstrated a rapid and profound effect on altering mitochondrial membrane potential (FIG. 6). C31 rapidly caused a drop in fluorescence emission in the red wavelength, indicating mitochondrial membrane depolarization and thus mitochondrial decompensation.

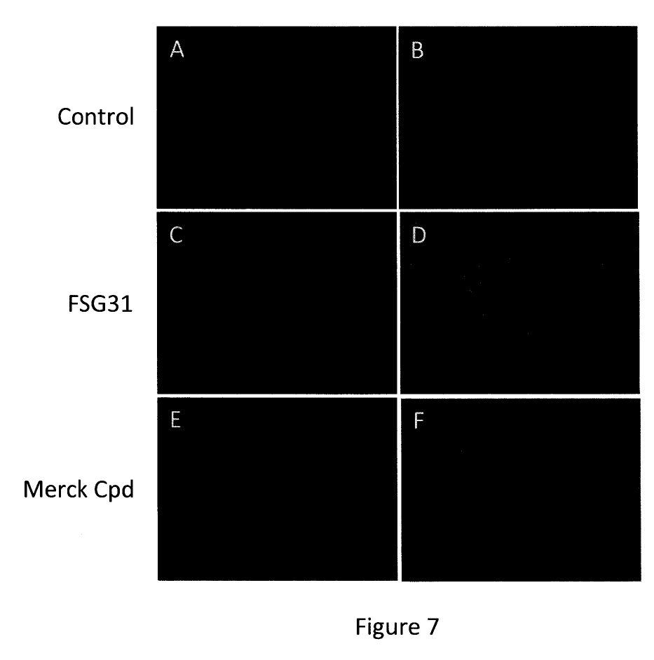

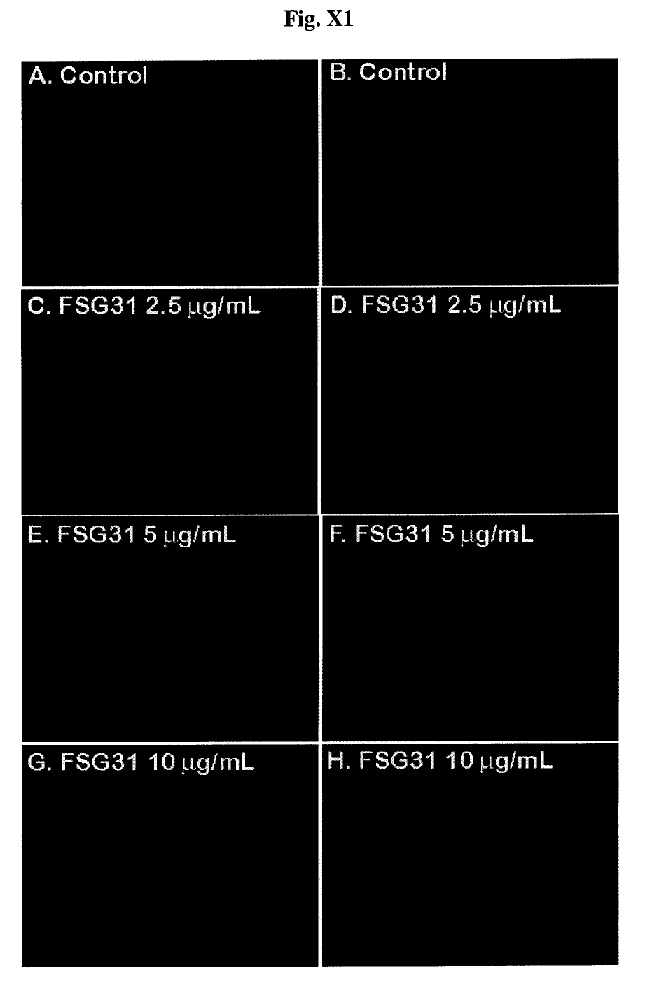

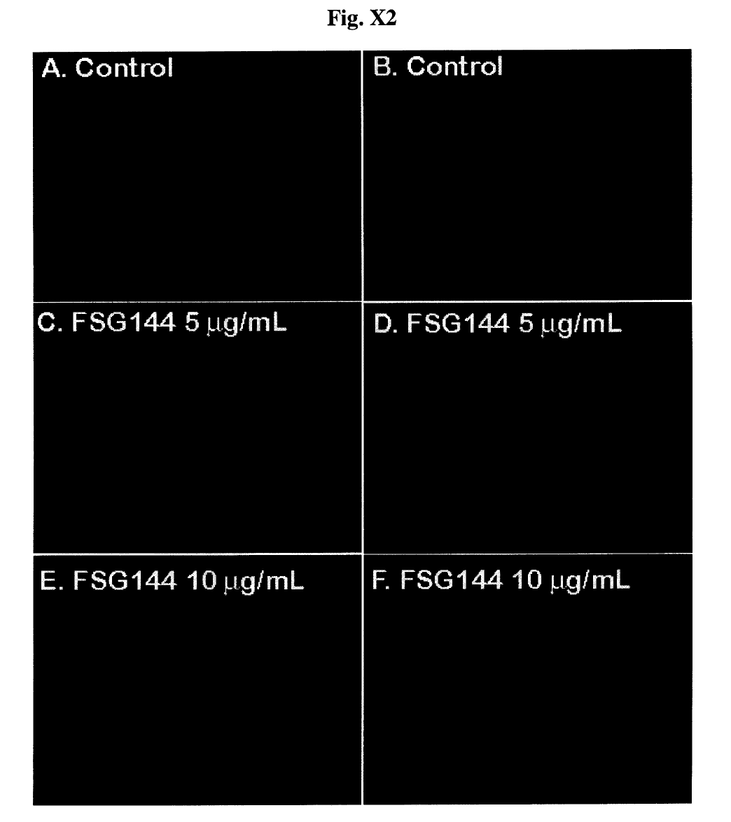

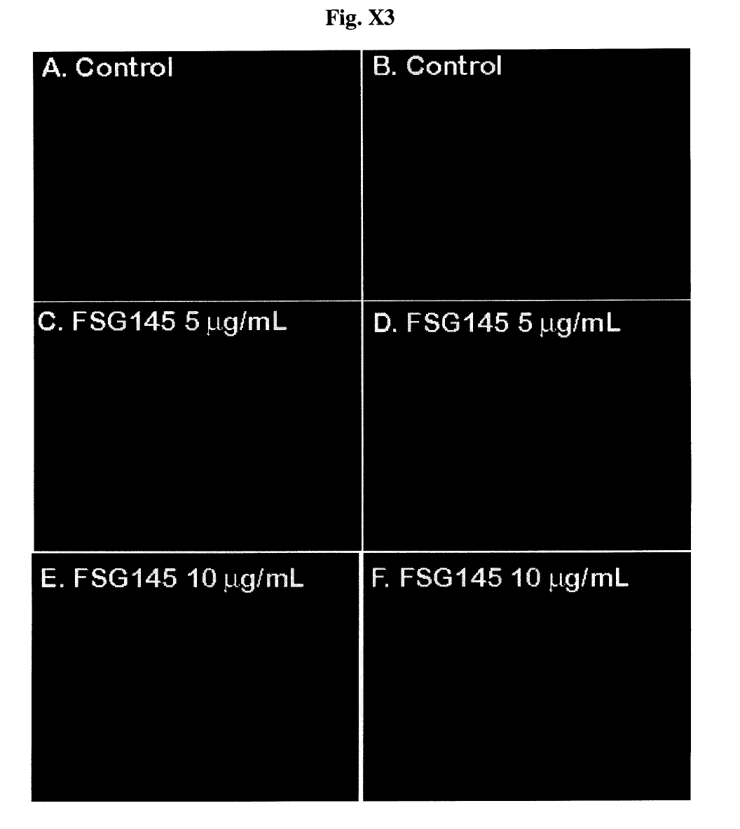

In contrast, Riv7d was 4 to 8 fold less effective. Riv7d failed to affect mitochondrial membrane potential and thus did not affect mitochondrial membrane potential a thus did not affect mitochondrial integrity. This can be visualized directly by capturing immunfluorescence micrographs (FIG. 7). Whereas C31 reduced red emission, vehicle control and Riv7d did not affect red fluorescence. Thus, C31, which inhibits mitochondrial FAS, affects mitochondrial function in cancer cells while Riv7d which does not inhibit mitochondrial FAS does not.

In an additional experiment Figure X1 shows JC-1 staining of SKOV3 cells in the presence of C31 (also referred to as FSG31). There is a depolarization of the mitochondria in the SKOV3 human ovarian cancer cells which is dose-dependent with C31 concentration. The left panels show granular red-staining indicating mitochondria with intact membrane potential is brightest in control, and decreases with increasing concentrations of C31. Conversely, the right panels show the diffuse cytoplasmic green-staining which indicates mitochondrial depolarization, is dimmest in control and brightest at the highest concentration of C31 tested. Drug was solubilized in DMSO and applied for 30 min prior to staining.

In another experiment Figure X2 shows JC-1 staining of SKOV3 cells in the presence of C144 (also referred to as FSG144). Similar to C31, there is a depolarization of the mitochondria in the SKOV3 human ovarian cancer cells which is dose-dependent with C144 concentration. The left panels show granular red-staining indicating mitochondria with intact membrane potential is brightest in control, and decreases with increasing concentrations of C144. Conversely, the right panels show the diffuse cytoplasmic green-staining which indicates mitochondrial depolarization, is dimmest in control and brightest at the highest concentration of C144 tested. Drug was solubilized in DMSO and applied for 30 min prior to staining.

In a further experiment Figure X3 shows JC-1 staining of SKOV3 cells in the presence of C145 (also referred to as FSG145). Similar to both C31 and C144, there is a depolarization of the mitochondria in the SKOV3 human ovarian cancer cells which is dose-dependent with C145 concentration. The left panels show granular red-staining indicating mitochondria with intact membrane potential is brightest in control, and decreases with increasing concentrations of C145. Conversely, the right panels show the diffuse cytoplasmic green-staining which indicates mitochondrial depolarization, is dimmest in control and brightest at the highest concentration of C145 tested. Drug was solubilized in DMSO and applied for 30 min prior to staining.

### Example 3

### Cytotoxic Effects on Tumors and Cancer Cell Lines

This example explores the effect of representative compounds on cell viability in cancer cell lines or tumor xenografts. To determine the efficacy of various test compounds in killing cancer cells in vitro, two assays were performed: (i) cytotoxicity measured with XTT and (ii) clonogenic assays. In addition, in vivo effects were tested using (iii) size reduction of tumor xenografts.

(i) Cytotoxicity Measured with XTT

Cytotoxicity can be easily measured using XTT assays, which provide an indication of mitochondrial activity. The XTT assay is a non-radioactive alternative for the [⁵¹Cr] release cytotoxicity assay. XTT is a tetrazolium salt that is reduced to a formazan dye only by metabolically active, viable cells. The reduction of XTT is measured spectrophotometrically as OD₄₉₀-OD₆₅₀. Thus, cells that are actively metabolizing will form reaction product when subjected to XTT measurement.

For this experiment, cells were cultured under standard culture conditions. MCF7 human breast cancer cells were cultured in DMEM containing 5 mM glucose medium with 10% fetal calf serum, 0.01 mg/ml insulin and 1% penicillin/streptomycin. To measure the cytotoxicity of specific compounds against cancer cells, 9×10³ MCF7 cells were plated per well in 96-well plates. Following overnight culture, the compounds, dissolved in DMSO, were added to the wells in 1 μl volume at specified concentrations. Vehicle controls were run for each experiment. Each condition was run in triplicate. After 24 to 72 h of incubation, cells were incubated for 4 hours with the XTT reagent as per manufacturer's instructions (Cell Proliferation Kit II (XTT) Roche Diagnostics, New Jersey). Plates were read at OD₄₉₀ on a PerkinElmer Victor 1420. Three wells containing the XTT reagent without cells served as the plate blank. XTT data were reported as OD₄₉₀.

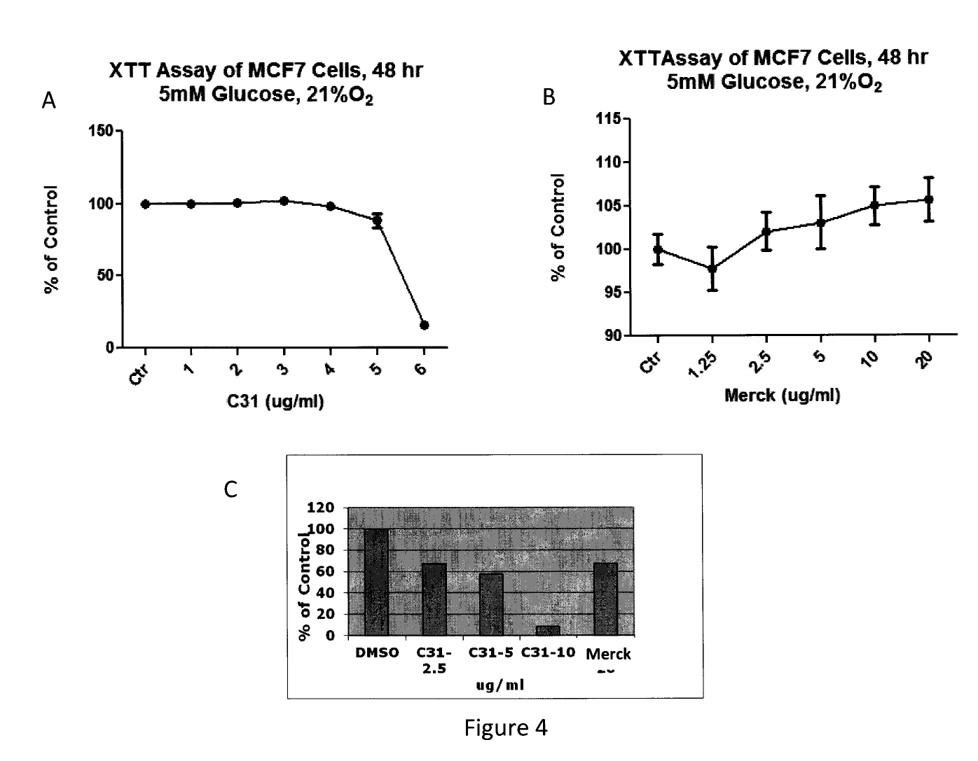

As seen in FIG. 4, C31 showed a concentration-dependent cytotoxicity (A), whereas Riv7d failed to affect cell viability (B) of MCF7 cells.

Using the XTT assay, LC₅₀ values for C31 were determined for numerous human cancer cell lines and human hepatocytes, as shown in the following table:

| | Number of Cell | LC₅₀ |
|---|---|---|
| Cell Line | Lines Tested | (μg/ml) |
| Human breast cancer | 5 | 3.3-16.7 |
| Human ovarian cancer | 2 | 6.0-8.1 |
| Human (non-small cell) lung cancer | 4 | 4.0-8.0 |
| Human colon cancer | 1 | 4 |
| Human hepatoma | 1 | 5 |
| Primary human hepatocytes | | 225 |

The LC₅₀'s for the human cancer cell lines range from 3.3-16.7 μg/mL (8.25-41.75 μM) compared to 225 μg/mL (562.5 μM) for primary human hepatocytes (non-malignant normal cells, determined using the alamarBlue® Assay which incorporates a fluorometric/colorimetric growth indicator based on detection of metabolic activity). This translates to a 34 to 170-fold potential therapeutic index in vitro for C31.

Using the XTT assay, LC₅₀ values for C144 were computed for numerous human cancer cell lines and human hepatocytes:

| Cell Line | LC₅₀ (μM) | IC₅₀ (μg/ml) |
|---|---|---|
| Human breast cancer (MCF-7) | 4.9 | 2.2 |
| Human ovarian cancer (OVCAR) | 9.0 | 4.0 |
| Human colon cancer (HCT116) | 10.8 | 4.8 |

Using the XTT assay, LC₅₀ values for C145 were computed for numerous human cancer cell lines and human hepatocytes:

| Cell Line | LC₅₀ (μM) | IC₅₀ (μg/ml) |
|---|---|---|
| Human breast cancer (MCF-7) | 3.9 | 1.8 |
| Human ovarian cancer (OVCAR) | 7.2 | 3.3 |
| Human colon cancer (HCT116) | 7.6 | 3.5 |

### (ii) Clonogenic Assays

In this assay, the effectiveness of specific agents on cell survival and proliferation can be measured in a functional sense. After treatment, surviving cells are re-plated to determine whether the surviving cells are still capable of proliferation. Therefore, this if a more direct measure of cell survival and function. It does not rely upon the function only of the mitochondria, but reflects the integrity and actual viability of the cell.

4×10⁵ cells were plated in 25 cm² flasks. After overnight culture, cells are treated with compounds for specified times and concentrations. After drug treatment, cells are trypsinized and equal numbers of treated and control cells are plated in triplicate in 60-mm dishes at a density of 500 cells/dish in medium without drugs. Clones are stained and counted after 7-10 days

This assay that directly measures cell viability is shown in FIG. 4(C). MCF 7 cells were treated with vehicle, C31 at increasing doses, or Riv7d. Whereas C31 caused a dose-dependent decrease in viability, Riv7d, even at a higher dose, failed to affect viability significantly.

(iii) Size Reduction of Tumor Xenografts.

Xenograft studies were performed using various mmFAS inhibitors disclosed herein. Human cancer xenograft studies are known in the art. (See Pizer et al., Cancer Res. 56:1189-1193, 1996; Pizer et al., Cancer Res. 60: 213-218, 2000; Testa et al., Oncogene 24: 7455-7464, 2005).

In particular, C31 mouse efficacy studies were conducted in order to evaluate anti-neoplastic activity of C31 in vivo. Athymic Nude mice were implanted with HCT116 human colon, SKOV3 human ovarian or OVCAR3 human ovarian cancer cell lines and were treated when the grown tumor xenografts reached at least 150 to 200 mm³.¹ ¹ Compounds were solubilized in TEP (80% polyethylene glycol (PEG) 400, 10% ethanol, 10% tween-80) in a volume of 50 μL. Mice were gavaged twice a day at doses indicated in the table.

| Study # | | | Average Tumor |
|---|---|---|---|
| (Route/# Days) | | Dose mg/kg | Size Endpoint |
| b.i.d. | Tumor | (# of animals) | (% Change) |
| 72406 | HCT-116 | Control (10) | +232 |
| (IP/8) | | 50 (11) | −59 |
| 3150772607 | SKOV-3 | Control (8) | +135 |
| (IP/10) | | 25 (8) | −74 |
| | | 50 (8) | −87 |
| | | 150 (8) | −83 |
| 121306 | SKOV-3 | Control (7) | +328 |
| (IP/12) | | 10 (7) | +179 |
| | | 50 (7) | −78 |
| | | 100 (7) | −78 |
| 11007 | SKOV-3 | Control (5) | +136 |
| (oral/11) | | 50 (6) | −78 |
| 1129063207 | OVCAR-3 | Control (4) | +252 |
| (oral/11) | | 50 (4) | −59 |
| 52307 | SKOV-3 | Control (8) | +376 |
| (Oral/15) | | 50 (8) | −75 |
| 101707 | HCT-116 | Control (5) | +2863 |
| (oral/16) | | FAS31 50 (5) | +256 |
| 3 Arms | | Control | +2863 |
| | | FAS144 53 (5) | −91 |
| | | Control | +2863 |
| | | FAS145 55 (5) | +236 |

The results demonstrated that C31 was cytotoxic to the HCT116 human colon, SKOV3 human ovarian, and OVCAR3 human ovarian cancer cell lines. The results also demonstrated that C31 was not cytotoxic to normal cells. The data from the various measures of cellular energy balance indicate that, compared to Riv7d (which is a high affinity FAS type I inhibitor), C31 (which has a lower affinity for cytoplasmic FAS I) is more effective at inhibition of mitochondrial lipid synthesis, depolarization of mitochondrial membrane potential, reduction in cellular ATP levels, activation of AMPK, and ultimately cytotoxicity of cancer cells as demonstrated by XTT and clonogenic assays. These data support the discovery that C31 is an inhibitor of mitochondrial fatty acid synthesis with the effect on cancer cells of inducing their metabolic compromise through disruption of mitochondria and compromising cellular energy levels in these cancer cells. In contrast, agents such as Riv7d, which are high affinity cytoplasmic FAS I inhibitors are ineffective at disruption of cancer cell metabolic pathways, energy compromise, and incur no cytotoxicity.

### Example 4

### Effects of Test Compounds on Cells in a Tumor-Like Environment

The microenvironment of most cancer cells involves a setting of relative hypoxia and hypoglycemia. It is well recognized that cancer cells have suborned and shifted normal metabolic pathways to advantage unregulated growth in such hostile environments. A surprising benefit of the present invention is that the therapy contemplated is particularly effective under these extreme metabolic conditions of hypoxia and hypoglycemia. This example duplicated many of the previous experiments using a variety of cancer cell lines, but culturing them under conditions of hypoxia, hypoglycemia, or a combination of hypoxia and hypoglycemia. This example explores the effect of representative compounds on various measures of cellular metabolism or energy balance: (i) cellular ATP level, (ii) AMP-activated protein kinase status, (iii) polarization of mitochondrial membranes, (iv) fatty acid synthesis, and (v) cytotoxicity in conditions mimicking a tumor-like environment.

(i) Cellular ATP level

Determination of the effect on cellular ATP level was carried out using the procedure described above. Cells were plated in 24 well cell culture plates at 2×10⁵ per well overnight. Cells were cultured in 1 mM or 5 mM glucose at either 1% or 21% (ambient) oxygen levels. These conditions were employed for treatment with vehicle, C31, or Riv7d. After drug treatment cells were lysed using 500 μl of TE buffer (4 mM Tris, 0.25 mM EDTA, pH 7.4) and boiled at 95° C. for 7 min. ATP levels were measured within the linear range using the ATP Bioluminescence Kit CLSII (Roche) by following the manufacture's recommendation. Luminescence was detected using PerkinElmer Victor 1420.

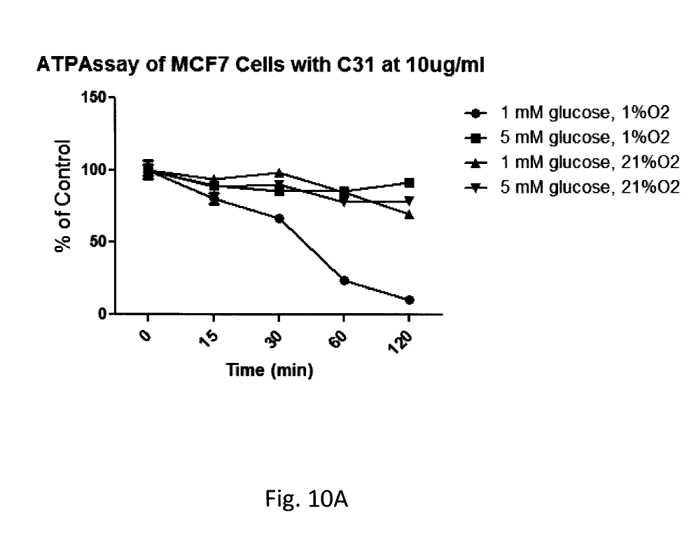

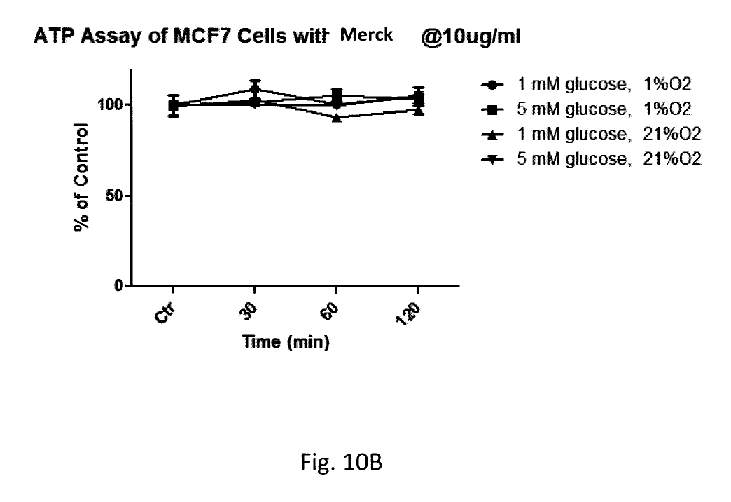

Under these conditions, ATP assays (FIGS. 10A, C31; FIG. 10B, Riv7d), were assessed. ATP levels declined in all of the cells treated with C31, especially when the cellular environment was both hypoxic and hypoglycemic. On the other hand, cells treated with Riv7d showed little effect on ATP levels.

(ii) AMP-activated Protein Kinase Status

Determination of AMP-activated protein kinase status was carried out using the procedure described above. Cells were grown in 6-well plates at 5×10⁴ per well and incubated overnight. Cells were cultured in 1 mM or 5 mM glucose at either 1% or 21% (ambient) oxygen levels. These conditions were employed for treatment with vehicle, C31, or Riv7d. Following drug treatment, the cells were harvested in Laemmli buffer and boiled for 5 minutes. Proteins were separated on a 4-15% SDS-PAGE gradient gel and transferred to a nitrocellulose membrane. Antibodies to pAMPK and AMPK were obtained from Cell Signaling.

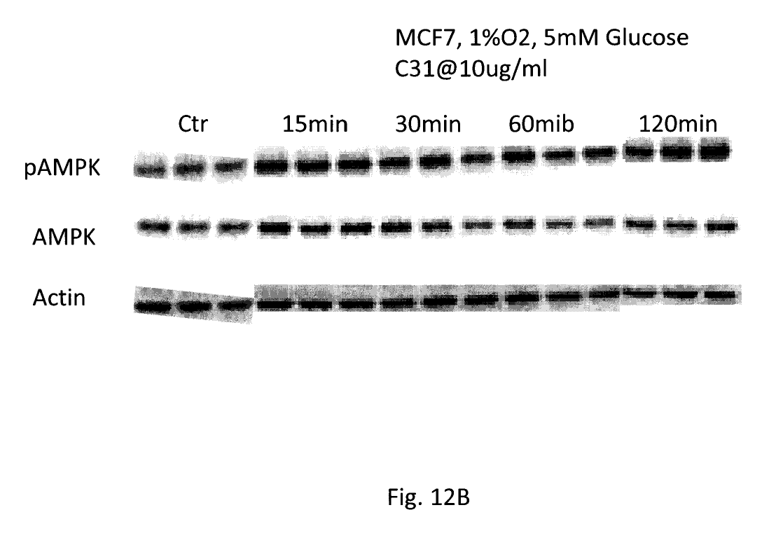

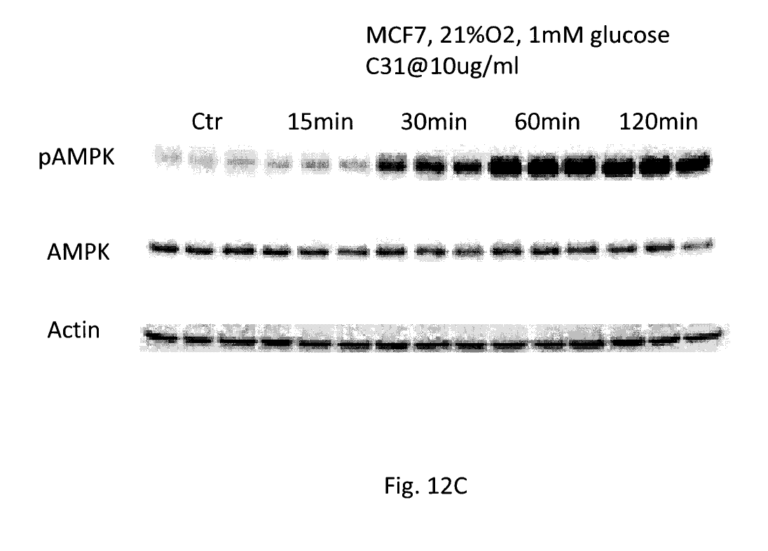

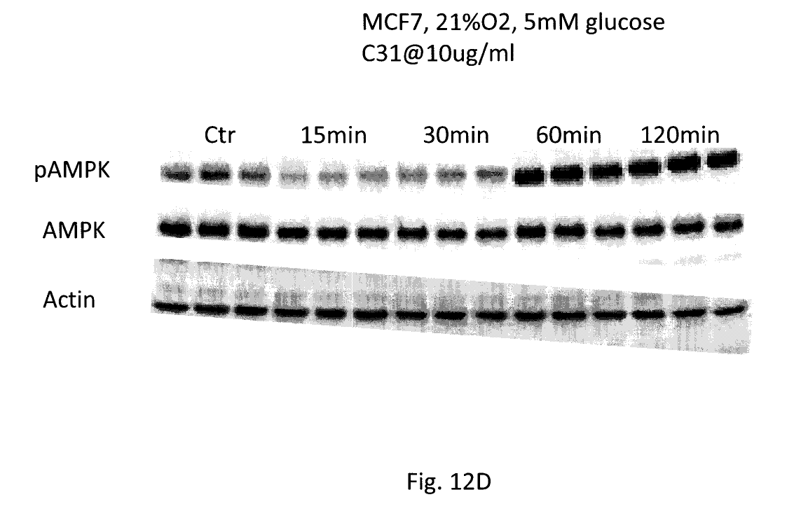

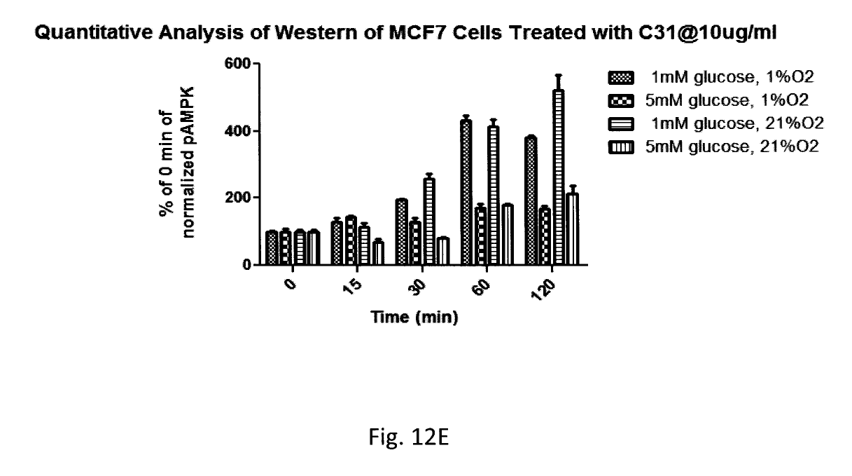

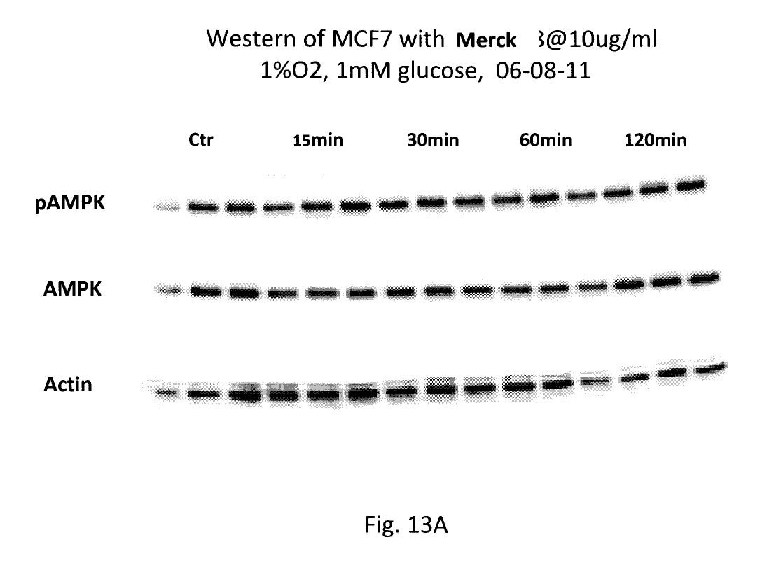

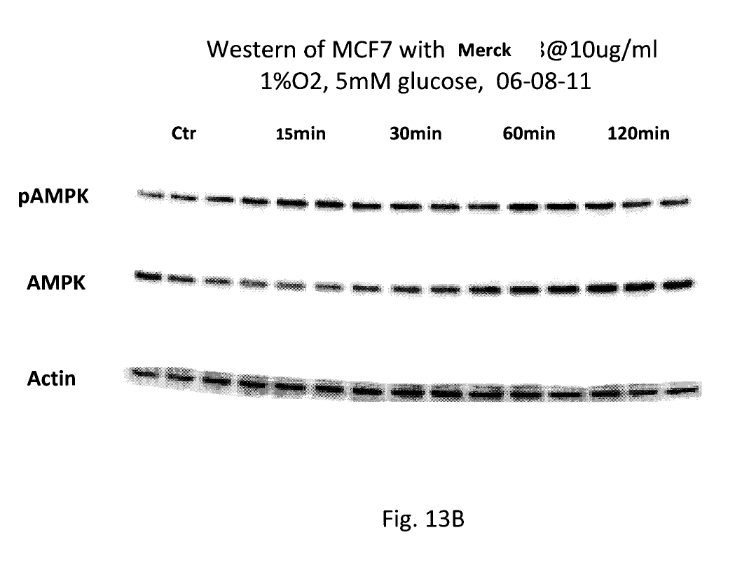

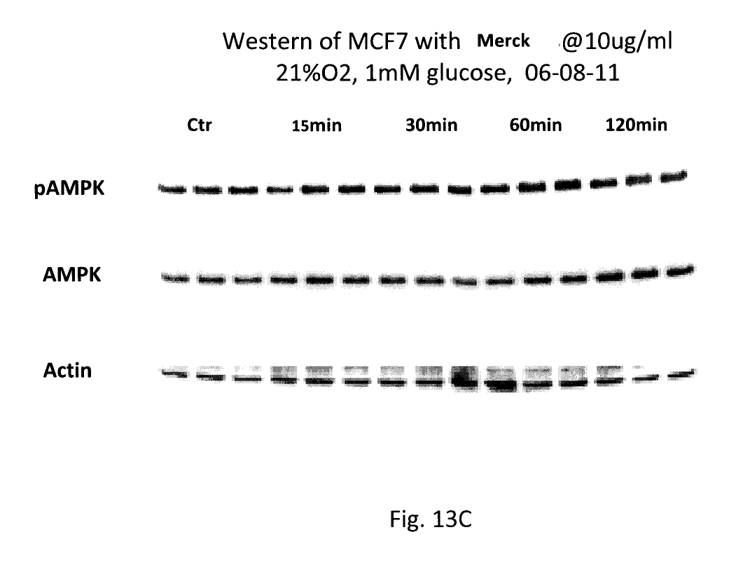

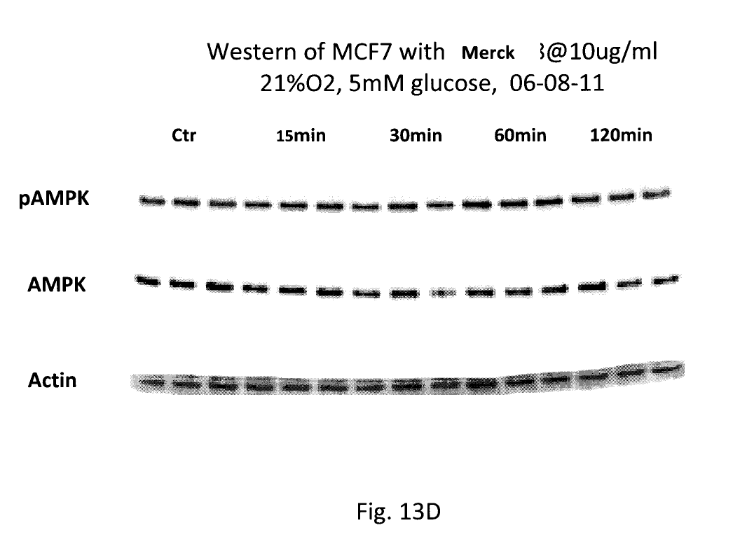

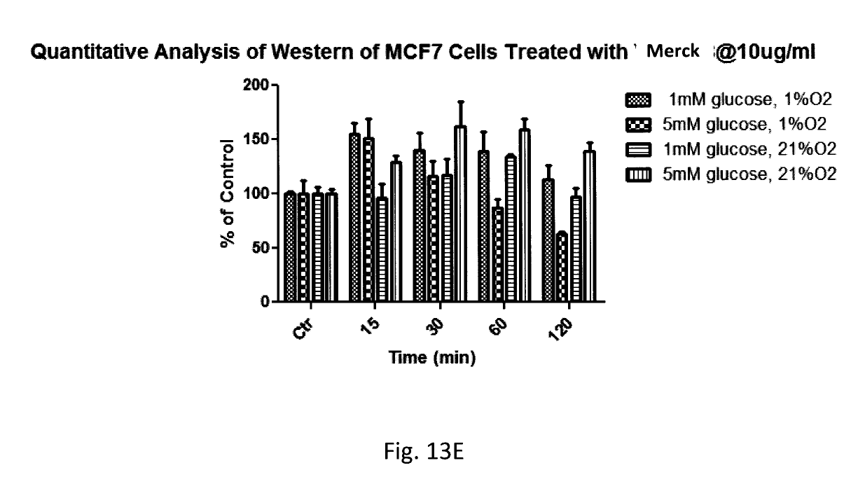

Under these conditions, AMPK activation (FIGS. 12A-E, C31; FIGS. 13 A-E, Riv7d) was assessed. Quantitative Western blots show increased phosphorylation of AMPK in cells treated with C31 (FIG. 12E), with substantially increased phosphorylation of AMPK under hypoglycemic conditions. Cells treated with Riv7d (FIG. 13E) do not show any particular trend.

(iii) Depolarization of Mitochondrial Membranes

Mitochondrial membrane polarization was monitored using the procedure described above. Cells were cultured in 24-well plates at 1×10⁴ per well and incubated overnight. Following drug inhibition the medium was removed and 1 μg/ml JC-1 (Molecular Probes) in DPBS was added to the cells. Plates were incubated for 30 min at 37° C., and washed two times with DPBS. Fluorescence from each well was measured at 530 nm/590 nm (excitation/emission) and by 485 nm/535 nm (excitation/emission) separately using PerkinElmer Victor 1420.

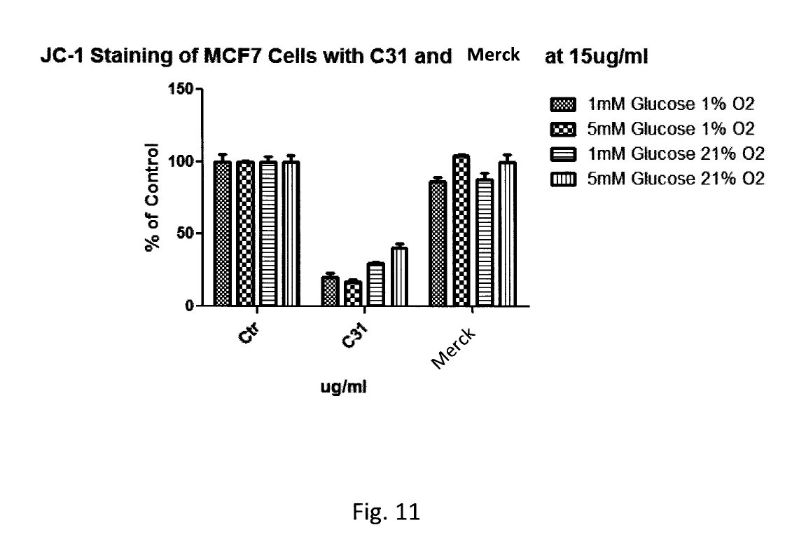

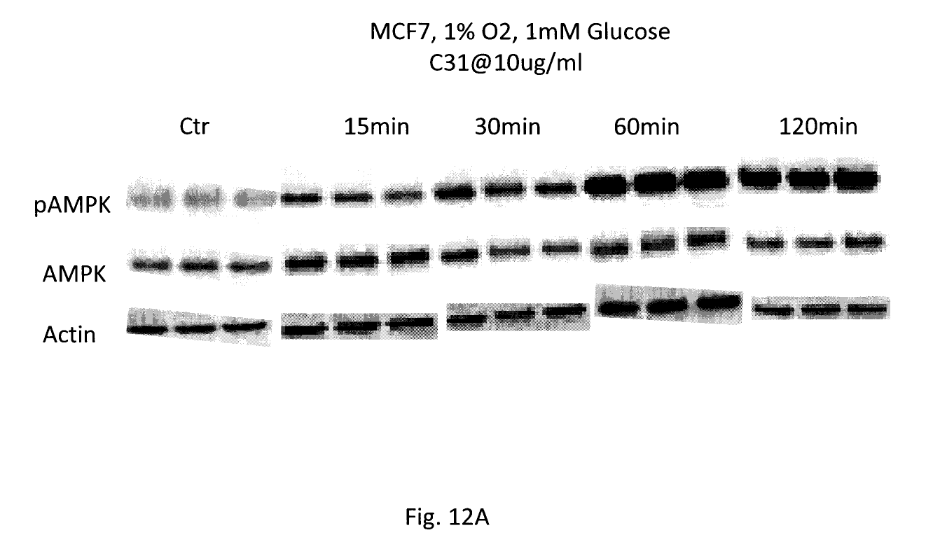

Under these conditions, JC-1 staining (FIG. 11) was assessed. The cells treated with C31 showed reductions in mitochondrial membrane polarization, which was exacerbated by hypoglycemia and particularly by hypoxia. Treatment with Riv7d had little effect on polarization, although a minor reduction was seen for cells under hypoglycemic conditions.

(iv) Fatty Acid Synthesis

MCF7 human breast cancer cells were cultured in DMEM containing 1 mM or 5 mM glucose in the medium with 10% fetal calf serum, 0.01 mg/ml insulin and 1% penicillin/streptomycin. The cells were grown in two oxygen conditions, normoxia (21% 02) or hypoxia (1% 02). For measurement of fatty acid synthesis cells were cultured in 24-well plates at 5×10⁴ per well and incubated overnight. Fatty acid synthesis was assayed by a 2 h pulse of [U¹⁴C]acetate, 1 μCi/well, followed by Folch extraction and scintillation counting with the addition of drugs as described. Controls were treated with DMSO alone.

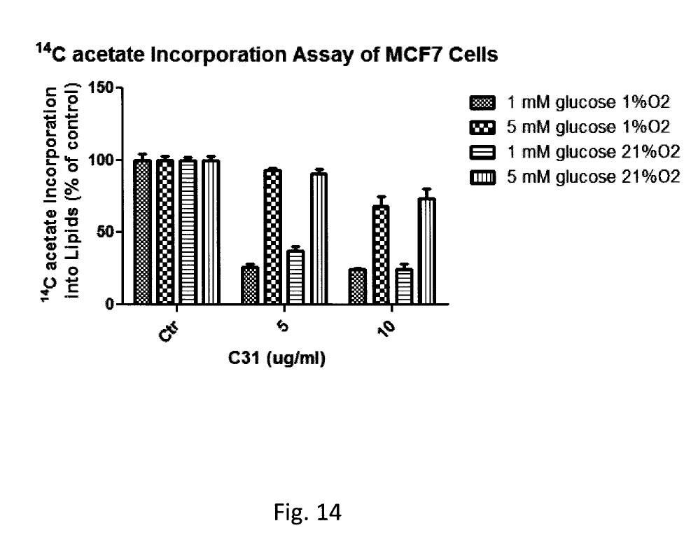

FIG. 14 shows that C31 is increasingly effective at inhibiting FAS activity in MCF7 cancer cells at low glucose and oxygen tension (hypoglycemia or hypoxia, respectively), which more accurately mimics the cancer microenvironment.

(v) Cytotoxicity

MCF7 human breast cancer cells were cultured in DMEM containing 1 mM or 5 mM glucose in the medium with 10% fetal calf serum, 0.01 mg/ml insulin and 1% penicillin/streptomycin. The cells were grown in two oxygen conditions, normoxia (21% 02) or hypoxia (1% 02). To measure the cytotoxicity of specific compounds against cancer cells, 9×10³ MCF7 cells were plated per well in 96-well plates. Following overnight culture, the compounds, dissolved in DMSO, were added to the wells in 1 μl volume at specified concentrations. After 24 to 72 h of incubation, cells were incubated for 4 h with the XTT reagent as per manufacturer's instructions (Cell Proliferation Kit II (XTT) Roche Diagnostics, New Jersey). Plates were read at OD₄₉₀ on a PerkinElmer Victor 1420. Three wells containing the XTT reagent without cells served as the plate blank. XTT data were reported as OD₄₉₀Vehicle controls were run for each experiment. Each condition was run in triplicate.

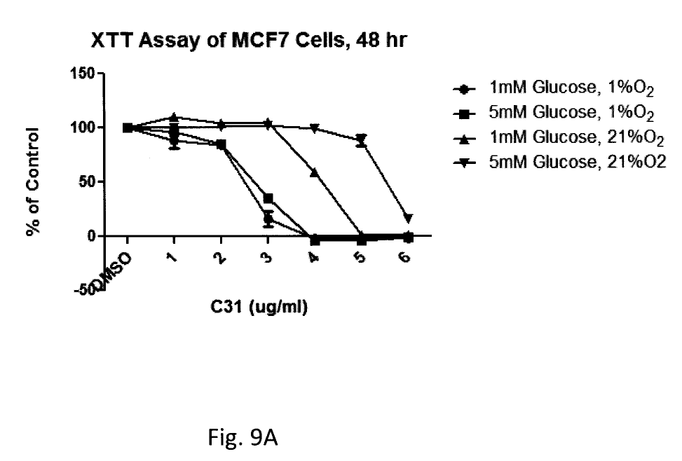

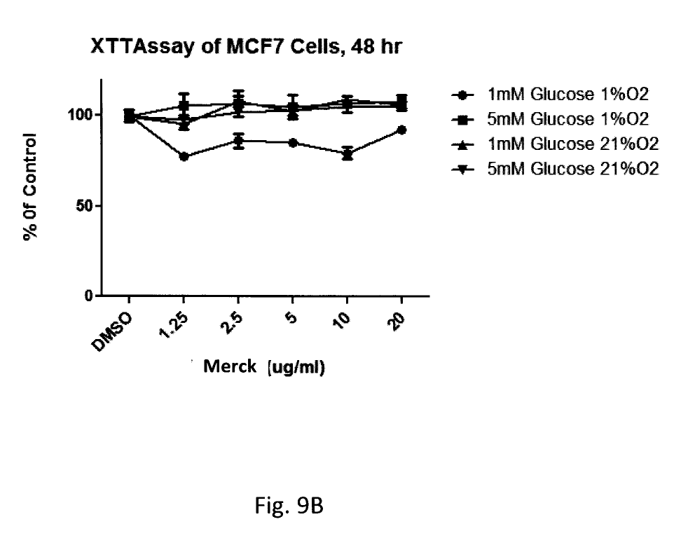

Under these conditions, XTT assays (FIGS. 9A, C31; FIG. 9B, Riv7d), were assessed. FIG. 9A shows that the cytotoxicity of C31 increases if the cells are hypoglycemic and even more if the cells are hypoxic. On the other hand, Riv7d in FIG. 9B shows no effect. (The lower cellular activity under conditions that are both hypoxic and hypoglycemic is independent of the concentration of the test compound).

Results:

Confirming that the mitochondrial metabolic pathways and mitochondrial fatty acid biosynthesis are critical to the survival of cancer cells in microenvironments often associated with cancer growth, C31 was even more effective under conditions of hypoxia and hypoglycemia than it was under standard culture conditions of 5 mM glucose and ambient oxygen. Furthermore, Riv7d was equally ineffective under conditions of hypoxia and hypoglycemia. In addition, the effectiveness of Riv7d was not enhanced by conditions of hypoxia and hypoglycemia, whereas C31 was. These data indicate that mitochondrial fatty acid biosynthesis plays a key role in the maintenance of metabolic integrity and homeostasis in cancer cells, and that this pathway is even more important, and thus a target of opportunity, in the hostile metabolic environment in which cancer cells grow.

C31 is much more effective as a FAS inhibitor under conditions commensurate with the cancer cell environment of hypoglycemia and hypoxia. This supports the invention that targeting mitochondrial FAS activities, which are relevant for cancer cell homeostasis. C31 is more effective at inducing metabolic compromise and cell death of MCF7 cancer cells at low glucose and oxygen tension (hypoglycemia or hypoxia, respectively), which more accurately mimics the cancer microenvironment, while Riv7d fails to demonstrate any activity in conditions of hypoglycemia or hypoxia.

The principles, preferred embodiments and modes of operation of the present invention have been described in the foregoing specification. The invention which is intended to be protected herein, however, is not to be construed as limited to the particular forms disclosed, since they are to be regarded as illustrative rather than restrictive. Variations and changes may be made by those skilled in the art without departing from the spirit of the invention.

All publications and patent applications mentioned in this specification are indicative of the level of skill of those skilled in the art to which this invention pertains. All publications and patent applications are herein incorporated by reference to the same extent as if each individual publication or patent application was specifically and individually indicated to be incorporated by reference.

### PARTIAL LIST OF DOCUMENTS CITED HEREIN

Abu-Elheiga, L., Brinkley, W. R., Zhong, L., Chirala, S. S., Woldegiorgis, G., and Wakil, S. J. (2000). The subcellular localization of acetyl-CoA carboxylase 2. Proc Natl Acad Sci USA 97, 1444-1449.
Autio, K. J., Kastaniotis, A. J., Pospiech, H., Miinalainen, I. J., Schonauer, M. S., Dieckmann, C. L., and Hiltunen, J. K. (2008). An ancient genetic link between vertebrate mitochondrial fatty acid synthesis and RNA processing. FASEB J 22, 569-578.
Bloch, K., and Vance, D. (1977). Control mechanisms in the synthesis of saturated fatty acids. Annu Rev Biochem 46, 263-298.
Brody, S., and Mikolajczyk, S. (1988). Neurospora mitochondria contain an acyl-carrier protein. Eur J Biochem 173, 353-359.
Brody, S., Oh, C., Hoja, U., and Schweizer, E. (1997). Mitochondrial acyl carrier protein is involved in lipoic acid synthesis in Saccharomyces cerevisiae. FEBS Lett 408, 217-220.
Challem, J. J. (1999). Toward a new definition of essential nutrients: is it now time for a third ‘vitamin’ paradigm? Med Hypotheses 52, 417-422.
Chen, Z., Kastaniotis, A. J., Miinalainen, I. J., Rajaram, V., Wierenga, R. K., and Hiltunen, J. K. (2009). 17beta-hydroxysteroid dehydrogenase type 8 and carbonyl reductase type 4 assemble as a ketoacyl reductase of human mitochondrial FAS. FASEB J 23, 3682-3691.
Cronan, J. E., Fearnley, I. M., and Walker, J. E. (2005). Mammalian mitochondria contain a soluble acyl carrier protein. FEBS Lett 579, 4892-4896.
Gabrielson, E. W., Pinn, M. L., Testa, J. R., and Kuhajda, F. P. (2001). Increased fatty acid synthase is a therapeutic target in mesothelioma. Clin Cancer Res 7, 153-157.
Harington, A., Herbert, C. J., Tung, B., Getz, G. S., and Slonimski, P. P. (1993). Identification of a new nuclear gene (CEM1) encoding a protein homologous to a beta-keto-acyl synthase which is essential for mitochondrial respiration in Saccharomyces cerevisiae. Mol Microbiol 9, 545-555.
Hiltunen, J. K., Autio, K. J., Schonauer, M. S., Kursu, V. A., Dieckmann, C. L., and Kastaniotis, A. J. (2010a). Mitochondrial fatty acid synthesis and respiration. Biochim Biophys Acta 1797, 1195-1202.
Hiltunen, J. K., Chen, Z., Haapalainen, A. M., Wierenga, R. K., and Kastaniotis, A. J. (2010b). Mitochondrial fatty acid synthesis—an adopted set of enzymes making a pathway of major importance for the cellular metabolism. Prog Lipid Res 49, 27-45.
Hiltunen, J. K., Schonauer, M. S., Autio, K. J., Mittelmeier, T. M., Kastaniotis, A. J., and Dieckmann, C. L. (2009). Mitochondrial fatty acid synthesis type II: more than just fatty acids. J Biol Chem 284, 9011-9015.
Hopwood, D. A., and Sherman, D. H. (1990). Molecular genetics of polyketides and its comparison to fatty acid biosynthesis. Annu Rev Genet 24, 37-66.
Jordan, S. W., and Cronan, J. E., Jr. (1997). A new metabolic link. The acyl carrier protein of lipid synthesis donates lipoic acid to the pyruvate dehydrogenase complex in Escherichia coli and mitochondria. J Biol Chem 272, 17903-17906.
Joshi, A. K., Zhang, L., Rangan, V. S., and Smith, S. (2003). Cloning, expression, and characterization of a human 4′-phosphopantetheinyl transferase with broad substrate specificity. J Biol Chem 278, 33142-33149.
Kuhajda, F. P. (2000). Fatty-acid synthase and human cancer: new perspectives on its role in tumor biology. Nutrition 16, 202-208.
Kuhajda, F. P. (2006). Fatty acid synthase and cancer: new application of an old pathway. Cancer Res 66, 5977-5980.
Kuhajda, F. P. (2008). AMP-activated protein kinase and human cancer: cancer metabolism revisited. Int J Obes (Lond) 32 Suppl 4, S36-41.
Kuhajda, F. P., Jenner, K., Wood, F. D., Hennigar, R. A., Jacobs, L. B., Dick, J. D., and Pasternack, G. R. (1994). Fatty acid synthesis: a potential selective target for antineoplastic therapy. Proc Natl Acad Sci USA 91, 6379-6383.
Lynen, F. (1980). On the structure of fatty acid synthetase of yeast. Eur J Biochem 112, 431-442.
Maczurek, A., Hager, K., Kenklies, M., Sharman, M., Martins, R., Engel, J., Carlson, D. A., and Munch, G. (2008). Lipoic acid as an anti-inflammatory and neuroprotective treatment for Alzheimer's disease. Adv Drug Deliv Rev 60, 1463-1470.
Pathania, D., Millard, M., and Neamati, N. (2009). Opportunities in discovery and delivery of anticancer drugs targeting mitochondria and cancer cell metabolism. Adv Drug Deliv Rev 61, 1250-1275.
Pepe, S., Leong, J. Y., Van der Merwe, J., Marasco, S. F., Hadj, A., Lymbury, R., Perkins, A., and Rosenfeldt, F. L.
(2008). Targeting oxidative stress in surgery: effects of ageing and therapy. Exp Gerontol 43, 653-657.
Pizer, E. S., Jackisch, C., Wood, F. D., Pasternack, G. R., Davidson, N. E., and Kuhajda, F. P. (1996a). Inhibition of fatty acid synthesis induces programmed cell death in human breast cancer cells. Cancer Res 56, 2745-2747.

1. A method for identifying compounds likely to inhibit growth of tumors, the method comprising:
(a) performing an assay that measures a biological activity in the presence and absence of a test compound; and
(b) determining whether the presence of the test compound inhibits the activity, wherein the assay measures an activity selected from the group consisting of activity of mmFAS, activity of Type I FAS, mitochondrial transmembrane potential, and cell viability.

2. (canceled)

3. The method of claim 1, wherein the activity of the mmFAS is measured in the mitochondria.

4. The method of claim 2, wherein the activity of the mmFAS is determined by rate of incorporation of malonate into lipids.

5. The method of claim 4, wherein the rate of incorporation is measured using radioactivity.

6. The method of claim 1, wherein the assay measures the activity of Type I FAS.

7. The method of claim 6, wherein Type I FAS activity is determined by rate of incorporation of acetate into lipids.

8. (canceled)

9. The method of claim 1, wherein the transmembrane potential is measured by transmembrane accumulation of a fluorescent dye.

10. (canceled)

11. The method of claim 1, further wherein a second activity from said group is measured in the presence and absence of said test compound.

12. The method of claim 1, wherein the test compound exhibits an IC₅₀ for inhibition of mmFAS that is lower than its IC₅₀ inhibition of Type I FAS activity.

13. The method of claim 12, wherein the ratio of IC₅₀ against Type I FAS is at least ten fold higher than the IC₅₀ for mmFAS.

14. The method according to claim 1, wherein the test compound reduces the growth of a xenograft tumor without killing the host.

15. (canceled)

16. A method of inhibiting cancer cell proliferation comprising exposing cancer cells to a compound which exhibits an IC₅₀ for inhibition of mammalian mitochondrial fatty acid synthase (mmFAS) that is lower than its IC₅₀ for inhibition of Type I fatty acid synthase (FAS) activity.

17. The method of claim 16, wherein the IC₅₀ for inhibition of Type I FAS is at least ten fold higher than the IC₅₀ for mmFAS.

18. The method of inhibiting cancer cell proliferation according to claim 16 comprising exposing cancer cells to an inhibitor of mmFAS in an amount sufficient to interrupt the metabolic balance of cells in the tumor and optionally wherein administration of said mmFAS inhibitor reduces cellular ATP level, reduces mitochondrial membrane polarization, and/or increases AMP-activated protein kinase phosphorylation.

19. (canceled)

20. The method of inhibiting cancer cell proliferation according to claim 16 comprising administering an inhibitor of mmFAS in an amount cytotoxic to tumor cells under restricted levels of oxygenation and/or nutrient levels.

21. The method of inhibiting cancer cell proliferation according to claim 16, comprising administering an inhibitor of mmFAS to a subject having a solid tumor in an amount sufficient to achieve systemic concentration of the inhibitor which is cytotoxic to cells on the outside of the tumor, whereby a lower concentration of the inhibitor on the interior of the tumor is cytotoxic to interior cells.

22. The method according to claim 16, wherein the mmFAS inhibitor has a structure represented by Formula I: 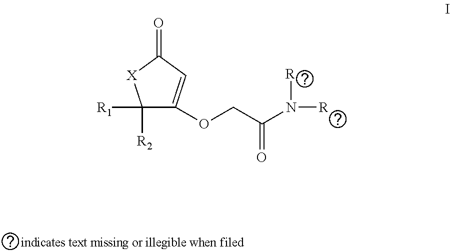
wherein X is comprised of a heteroatom which may be selected from any one of O, S, or NR, where R is H, alkyl, alkenyl, aryl, arylaklyl, or alkylaryl, R₁ and R₂ are independently selected from H, C1-C20 alkyl, cycloalkyl, alkenyl, aryl, arylalkyl, or alkylaryl, and R₃ and R₄ are independently either a hydrogen atom or are members of a substituted or unsubstituted ring having 4-6 carbon atoms, or where if neither R3 and R4 is a hydrogen, then they together form an optionally substituted ring structure having 4-6 carbon atoms.

23. The method according to claim 22, wherein the fatty acid synthase inhibitor is a compound having the formula:

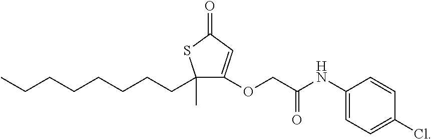 [SMILES: CCCCCCCCC1(C)SC(=O)C=C1OCC(=O)Nc1ccc(Cl)cc1]

24. The method according to claim 16, wherein the mmFAS inhibitor is a diphenyl ether.

25. (canceled)

26. The method according to claim 22, wherein the cancer cell is a pre-cancerous or cancerous lesion in a tissue type in human selected from the group consisting of breast, prostate, colon, lung, stomach, mouth, bile duct, ovarian, brain, or liver.

27. A method for determining the responsiveness of a pre-cancerous or cancerous lesion to mitochondrial FAS inhibitors using serum testing or tissue immunohistochemistry to evaluate for the presence of mitochondrial FAS, or by assaying tumor tissue directly for its sensitivity against mitochondrial FAS inhibitors, or by assaying for over-expression of mitochondrial FAS, either in the tumor tissue directly or in the tumor host.

28. (canceled)

29. (canceled)
